SEQUENCE LISTING (185 sequences)

SEQ ID NO: 1 - protein, 28 aa, Artificial
  Artificial peptide derived from HIV-1
RVIEVVQGACRAIRHIPRRIRQGLERIL

SEQ ID NO: 2 - protein, 21 aa, Artificial
  Artificial peptide derived from HIV-1
YHRLRDLLLIVTRIVELLGRR

SEQ ID NO: 3 - protein, 28 aa, Artificial
  Artificial peptide derived from HIV-1
DLWETLRRGGRWILAIPRRIRQGLELTL

SEQ ID NO: 4 - protein, 25 aa, Artificial
  Artificial peptide derived from HIV-1
FLIRQLIRLLTWLFSNCRTLLSRVY

SEQ ID NO: 5 - protein, 28 aa, Artificial
  Artificial peptide derived from HIV-1
LLSRVYQILQPILQRLSATLQRIREVLR

SEQ ID NO: 6 - protein, 29 aa, Artificial
  Artificial peptide derived from HIV-1
RIAGYGLRGLAVIIRICIRGLNLIFEIIR

SEQ ID NO: 7 - protein, 21 aa, Artificial
  Artificial peptide derived from HIV-1
RVIEVVQGACRAIRHIPRRIR

SEQ ID NO: 8 - protein, 17 aa, Artificial
  Artificial peptide derived from HIV-1
VVQGACRAIRHIPRRIR

SEQ ID NO: 9 - protein, 14 aa, Artificial
  Artificial peptide derived from HIV-1
GACRAIRHIPRRIR

SEQ ID NO: 10 - protein, 21 aa, Artificial
  Artificial peptide derived from HIV-1
RVIRVVQGACRAIRHIPRRIR

SEQ ID NO: 11 - protein, 21 aa, Artificial
  Artificial peptide derived from HIV-1
RVIEVVRGACRAIRHIPRRIR

SEQ ID NO: 12 - protein, 21 aa, Artificial
  Artificial peptide derived from HIV-1
RVIEVVQGICRAIRHIPRRIR

SEQ ID NO: 13 - protein, 21 aa, Artificial
  Artificial peptide derived from HIV-1
RVISVVQGACRAIRRIPRRIR

SEQ ID NO: 14 - protein, 28 aa, Artificial
  Artificial peptide derived from HIV-1
RVIRVVQGACRAIRHIPRRIRQGLERIL

SEQ ID NO: 15 - protein, 28 aa, Artificial
  Artificial peptide derived from HIV-1
RVIEVVRGACRAIRHIPRRIRQGLERIL

SEQ ID NO: 16 - protein, 28 aa, Artificial
  Artificial peptide derived from HIV-1
RVIEVVQGICRAIRHIPRRIRQGLERIL

SEQ ID NO: 17 - protein, 28 aa, Artificial
  Artificial peptide derived from HIV-1
RVISVVQGACRAIRRIPRRIRQGLERIL

SEQ ID NO: 18 - protein, 28 aa, Artificial
  Artificial peptide derived from HIV-1
RVIEVVQGACRAIRHIPRRIRQILERIL

SEQ ID NO: 19 - protein, 28 aa, Artificial
  Artificial peptide derived from HIV-1
RVIEVVQGACRAIRHIPRRIRQGLRRIL

SEQ ID NO: 20 - protein, 21 aa, Artificial
  Artificial peptide derived from HIV-1
RVIRVVQGACRAIRHIPRRIR

SEQ ID NO: 21 - protein, 21 aa, Artificial
  Artificial peptide derived from HIV-1
RVIEVVRGACRAIRHIPRRIR

SEQ ID NO: 22 - protein, 21 aa, Artificial
  Artificial peptide derived from HIV-1
RVIEVVQGICRAIRHIPRRIR

SEQ ID NO: 23 - protein, 21 aa, Artificial
  Artificial peptide derived from HIV-1
RVIEVVQGACRAIRRIPRRIR

SEQ ID NO: 24 - protein, 28 aa, Artificial
  Artificial peptide derived from HIV-1
RVIRVVQGACRAIRHIPRRIRQGLERIL

SEQ ID NO: 25 - protein, 28 aa, Artificial
  Artificial peptide derived from HIV-1
RVIEVVRGACRAIRHIPRRIRQGLERIL

SEQ ID NO: 26 - protein, 28 aa, Artificial
  Artificial peptide derived from HIV-1
RVIEVVQGICRAIRHIPRRIRQGLERIL

SEQ ID NO: 27 - protein, 28 aa, Artificial
  Artificial peptide derived from HIV-1
RVIEVVQGACRAIRRIPRRIRQGLERIL

SEQ ID NO: 28 - protein, 17 aa, Artificial
  Artificial peptide derived from HIV-1
VVRGACRAIRHIPRRIR

SEQ ID NO: 29 - protein, 17 aa, Artificial
  Artificial peptide derived from HIV-1
VVQGICRAIRHIPRRIR

SEQ ID NO: 30 - protein, 17 aa, Artificial
  Artificial peptide derived from HIV-1
VVQRACRAIRRIPRRIR

SEQ ID NO: 31 - protein, 23 aa, Artificial
  Artificial peptide derived from HIV-1
VVRGACRAIRHIPRRIRGLERIL

SEQ ID NO: 32 - protein, 23 aa, Artificial
  Artificial peptide derived from HIV-1
VVQGICRAIRHIPRRIRGLERIL

SEQ ID NO: 33 - protein, 23 aa, Artificial
  Artificial peptide derived from HIV-1
VVQGACRAIRRIPRRIRGLERIL

SEQ ID NO: 34 - protein, 14 aa, Artificial
  Artificial peptide derived from HIV-1
GACRAIRRIPRRIR

SEQ ID NO: 35 - protein, 20 aa, Artificial
  Artificial peptide derived from HIV-1
GACRAIRRIPRRIRGLERIL

SEQ ID NO: 36 - protein, 17 aa, Artificial
  Artificial peptide derived from HIV-1
VVQRACRAIRHIPRRIR

SEQ ID NO: 37 - protein, 14 aa, Artificial
  Artificial peptide derived from HIV-1
RACRAIRHIPRRIR

SEQ ID NO: 38 - protein, 21 aa, Artificial
  Artificial peptide derived from HIV-1
RVIRVVRGACRAIRHIPRRIR

SEQ ID NO: 39 - protein, 21 aa, Artificial
  Artificial peptide derived from HIV-1
RVIRVVRGACRAIRHIPRRIR

SEQ ID NO: 40 - protein, 17 aa, Artificial
  Artificial peptide derived from HIV-1
RRIRHIPRAIRVVQGAC

SEQ ID NO: 41 - protein, 21 aa, Artificial
  Artificial peptide derived from HIV-1
RIRRPIHRIARCAGQVVEIVR

SEQ ID NO: 42 - protein, 28 aa, Artificial
  Artificial peptide derived from HIV-1
LIRELGQRIRRPIHRIARCAGQVVEIVR

SEQ ID NO: 43 - protein, 28 aa, Artificial
  Artificial peptide derived from HIV-1
LIRELGQRIRRPIHRIARCAGQVVRIVR

SEQ ID NO: 44 - protein, 28 aa, Artificial
  Artificial peptide derived from HIV-1
LIRELGQRIRRPIHRIARCAGRVVEIVR

SEQ ID NO: 45 - protein, 28 aa, Artificial
  Artificial peptide derived from HIV-1
LIRELGQRIRRPIHRIARCIGQVVEIVR

SEQ ID NO: 46 - protein, 28 aa, Artificial
  Artificial peptide derived from HIV-1
LIRELGQRIRRPIRRIARCAGQVVEIVR

SEQ ID NO: 47 - protein, 28 aa, Artificial
  Artificial peptide derived from HIV-1
LIRELGIRIRRPIHRIARCAGQVVEIVR

SEQ ID NO: 48 - protein, 28 aa, Artificial
  Artificial peptide derived from HIV-1
LIRRLGQRIRRPIHRIARCAGQVVEIVR

SEQ ID NO: 49 - protein, 21 aa, Artificial
  Artificial peptide derived from HIV-1
RIRRPIHRIARCAGQVVEIVR

SEQ ID NO: 50 - protein, 21 aa, Artificial
  Artificial peptide derived from HIV-1
RIRRPIHRIARCAGQVVRIVR

SEQ ID NO: 51 - protein, 21 aa, Artificial
  Artificial peptide derived from HIV-1
RIRRPIHRIARCAGRVVEIVR

SEQ ID NO: 52 - protein, 21 aa, Artificial
  Artificial peptide derived from HIV-1
RIRRPIHRIARCIGQVVEIVR

SEQ ID NO: 53 - protein, 21 aa, Artificial
  Artificial peptide derived from HIV-1
RIRRPIRRIARCAGQVVEIVR

SEQ ID NO: 54 - protein, 21 aa, Artificial
  Artificial peptide derived from HIV-1
RIRRPIHRIIRCIGQVVRIVR

SEQ ID NO: 55 - protein, 21 aa, Artificial
  Artificial peptide derived from HIV-1
RIRRPIRRIIRCIGQVVEIVR

SEQ ID NO: 56 - protein, 24 aa, Artificial
  Artificial peptide derived from HIV-1
LIRELGQRIRRPIHRIARCAGQVV

SEQ ID NO: 57 - protein, 24 aa, Artificial
  Artificial peptide derived from HIV-1
LIRELRQRIRRPIHRIARCARQVV

SEQ ID NO: 58 - protein, 24 aa, Artificial
  Artificial peptide derived from HIV-1
LIRELGQRIRRPIHRIARCAGRVV

SEQ ID NO: 59 - protein, 24 aa, Artificial
  Artificial peptide derived from HIV-1
LIRELGQRIRRPIHRIARCIGQVV

SEQ ID NO: 60 - protein, 24 aa, Artificial
  Artificial peptide derived from HIV-1
LIRELGQRIRRPIRRIARCAGQVV

SEQ ID NO: 61 - protein, 24 aa, Artificial
  Artificial peptide derived from HIV-1
LIRELGIRIRRPIHRIARCAGQVV

SEQ ID NO: 62 - protein, 24 aa, Artificial
  Artificial peptide derived from HIV-1
LIRRLGQRIRRPIHRIARCAGQVV

SEQ ID NO: 63 - protein, 21 aa, Artificial
  Artificial peptide derived from HIV-1
LIRELGQRIRRPIHRIARCAG

SEQ ID NO: 64 - protein, 21 aa, Artificial
  Artificial peptide derived from HIV-1
LIRELGQRIRRPIHRIARCAR

SEQ ID NO: 65 - protein, 21 aa, Artificial
  Artificial peptide derived from HIV-1
LIRELGQRIRRPIHRIARCAI

SEQ ID NO: 66 - protein, 21 aa, Artificial
  Artificial peptide derived from HIV-1
LIRELGQRIRRPIHRIARCIG

SEQ ID NO: 67 - protein, 21 aa, Artificial
  Artificial peptide derived from HIV-1
LIRELGQRIRRPIRRIARCAG

SEQ ID NO: 68 - protein, 21 aa, Artificial
  Artificial peptide derived from HIV-1
LIRELGIRIRRPIHRIARCAG

SEQ ID NO: 69 - protein, 21 aa, Artificial
  Artificial peptide derived from HIV-1
LIRRLGQRIRRPIHRIARCAG

SEQ ID NO: 70 - protein, 15 aa, Artificial
  Artificial peptide derived from HIV-1
RAIRRAIRGAPRAIL

SEQ ID NO: 71 - protein, 19 aa, Artificial
  Artificial peptide derived from HIV-1
RAIRRAIRGAPRAILRAIL

SEQ ID NO: 72 - protein, 28 aa, Artificial
  Artificial peptide derived from HIV-1
KVIEVVQGACKAIKHIPKKIKQGLEKIL

SEQ ID NO: 73 - protein, 20 aa, Artificial
  Artificial peptide derived from HIV-1
LWETLRRGGRWILAIPRRIR

SEQ ID NO: 74 - protein, 28 aa, Artificial
  Artificial peptide derived from HIV-1
DLWETLRRIIRWILAIPRRIRQGLELTL

SEQ ID NO: 75 - protein, 28 aa, Artificial
  Artificial peptide derived from HIV-1
DLWETLRRGGRWILAIPRRIRQGLQLCL

SEQ ID NO: 76 - protein, 28 aa, Artificial
  Artificial peptide derived from HIV-1
DLWETLRRGCRWILAIPRRIRQGLELTL

SEQ ID NO: 77 - protein, 28 aa, Artificial
  Artificial peptide derived from HIV-1
DLWETLRRIIRWILAIPRRIRQGLELCL

SEQ ID NO: 78 - protein, 27 aa, Artificial
  Artificial peptide derived from HIV-1
LWETLRRGGRWILAIPRRIRQGLELTL

SEQ ID NO: 79 - protein, 27 aa, Artificial
  Artificial peptide derived from HIV-1
LWETLRRGGRWILAIPRRIRQGLELCL

SEQ ID NO: 80 - protein, 27 aa, Artificial
  Artificial peptide derived from HIV-1
LWETLRRGCRWILAIPRRIRQGLELTL

SEQ ID NO: 81 - protein, 27 aa, Artificial
  Artificial peptide derived from HIV-1
LWRTLRRGGRWILAIPRRIRQGLELTL

SEQ ID NO: 82 - protein, 27 aa, Artificial
  Artificial peptide derived from HIV-1
LWETLRRGGRWILAIPRRIRQGLRLTL

SEQ ID NO: 83 - protein, 27 aa, Artificial
  Artificial peptide derived from HIV-1
LWETLRRGGRWILAIPRRIRRGLELTL

SEQ ID NO: 84 - protein, 27 aa, Artificial
  Artificial peptide derived from HIV-1
LWETLRRGGRWILAIPRRIRRQIELTL

SEQ ID NO: 85 - protein, 27 aa, Artificial
  Artificial peptide derived from HIV-1
LWELLRRGGRWILAIPRRIRQGLELTL

SEQ ID NO: 86 - protein, 27 aa, Artificial
  Artificial peptide derived from HIV-1
LWRLLRRGGRWILAIPRRIRQGLELTL

SEQ ID NO: 87 - protein, 21 aa, Artificial
  Artificial peptide derived from HIV-1
DLWETLRRIIRWILAIPRRIR

SEQ ID NO: 88 - protein, 21 aa, Artificial
  Artificial peptide derived from HIV-1
DLWETLRRGGRWILAIPRRIR

SEQ ID NO: 89 - protein, 21 aa, Artificial
  Artificial peptide derived from HIV-1
DLWETLRRGCRWILAIPRRIR

SEQ ID NO: 90 - protein, 20 aa, Artificial
  Artificial peptide derived from HIV-1
LWETLRRGGRWILAIPRRIR

SEQ ID NO: 91 - protein, 20 aa, Artificial
  Artificial peptide derived from HIV-1
LWETLRRIIRWILAIPRRIR

SEQ ID NO: 92 - protein, 20 aa, Artificial
  Artificial peptide derived from HIV-1
LWETLRRGCRWILAIPRRIR

SEQ ID NO: 93 - protein, 20 aa, Artificial
  Artificial peptide derived from HIV-1
LWETLRRGGRWILAIPRRIR

SEQ ID NO: 94 - protein, 20 aa, Artificial
  Artificial peptide derived from HIV-1
LWETLRRGCRWILAIPRRIR

SEQ ID NO: 95 - protein, 20 aa, Artificial
  Artificial peptide derived from HIV-1
LWETLRRIIRWILAIPRRIR

SEQ ID NO: 96 - protein, 20 aa, Artificial
  Artificial peptide derived from HIV-1
LWELLRRGGRWILAIPRRIR

SEQ ID NO: 97 - protein, 20 aa, Artificial
  Artificial peptide derived from HIV-1
LWRLLRRGGRWILAIPRRIR

SEQ ID NO: 98 - protein, 16 aa, Artificial
  Artificial peptide derived from HIV-1
LRRGGRWILAIPRRIR

SEQ ID NO: 99 - protein, 20 aa, Artificial
  Artificial peptide derived from HIV-1
LWETLRRGGRWILAIPRAIL

SEQ ID NO: 100 - protein, 16 aa, Artificial
  Artificial peptide derived from HIV-1
LRRGGRWILAIPRAIL

SEQ ID NO: 101 - protein, 20 aa, Artificial
  Artificial peptide derived from HIV-1
LWETLRRGGRWILAIPRAIL

SEQ ID NO: 102 - protein, 16 aa, Artificial
  Artificial peptide derived from HIV-1
LRRGGRWILAIPREIL

SEQ ID NO: 103 - protein, 18 aa, Artificial
  Artificial peptide derived from HIV-1
WILAIPRRIRGGRLWETL

SEQ ID NO: 104 - protein, 18 aa, Artificial
  Artificial peptide derived from HIV-1
WETLPRRIRGGRLWILAI

SEQ ID NO: 105 - protein, 20 aa, Artificial
  Artificial peptide derived from HIV-1
RIRRPIALIWRGGRRLTEWL

SEQ ID NO: 106 - protein, 28 aa, Artificial
  Artificial peptide derived from HIV-1
DLWETLKKGGRWILAIPRRIKQGLELTL

SEQ ID NO: 107 - protein, 27 aa, Artificial
  Artificial peptide derived from HIV-1
LWETLGRVGRWVLAIPRRIRQGLELAL

SEQ ID NO: 108 - protein, 21 aa, Artificial
  Artificial peptide derived from HIV-1
YHRLRRLLLIVTRIVELLGRR

SEQ ID NO: 109 - protein, 21 aa, Artificial
  Artificial peptide derived from HIV-1
YHRLRDLLRIVTRIVELLGRR

SEQ ID NO: 110 - protein, 21 aa, Artificial
  Artificial peptide derived from HIV-1
YHRLRDLLLIVRRIVELLGRR

SEQ ID NO: 111 - protein, 21 aa, Artificial
  Artificial Peptide derived from HIV-1
YHRLRDLLLIVTRIVRLLGRR

SEQ ID NO: 112 - protein, 21 aa, Artificial
  Artificial Peptide derived from HIV-1
YHRLRDLLLIVTRIVCLLGRR

SEQ ID NO: 113 - protein, 21 aa, Artificial
  Artificial Peptide derived from HIV-1
YHRLRDLLLIVRRIVCLLGRR

SEQ ID NO: 114 - protein, 21 aa, Artificial
  Artificial Peptide derived from HIV-1
YHRLLRDLLIVTRIVELLGRR

SEQ ID NO: 115 - protein, 18 aa, Artificial
  Artificial Peptide derived from HIV-1
YHRLRRLLLIVTRIVELL

SEQ ID NO: 116 - protein, 18 aa, Artificial
  Artificial Peptide derived from HIV-1
YHRLRDLLRIVTRIVELL

SEQ ID NO: 117 - protein, 18 aa, Artificial
  Artificial Peptide derived from HIV-1
YHRLRDLLLIVRRIVELL

SEQ ID NO: 118 - protein, 18 aa, Artificial
  Artificial Peptide derived from HIV-1
YHRLRDLLLIVTRIVRLL

SEQ ID NO: 119 - protein, 18 aa, Artificial
  Artificial Peptide derived from HIV-1
YHRLRDLLLIVTRIVCLL

SEQ ID NO: 120 - protein, 18 aa, Artificial
  Artificial Peptide derived from HIV-1
THRLRDLLLIVRRIVCLL

SEQ ID NO: 121 - protein, 18 aa, Artificial
  Artificial Peptide derived from HIV-1
YHRLLRDLLIVTRIVELL

SEQ ID NO: 122 - protein, 18 aa, Artificial
  Artificial Peptide derived from HIV-1
YHRLRDLLLIVTRIVELL

SEQ ID NO: 123 - protein, 21 aa, Artificial
  Artificial Peptide derived from HIV-1
RRGLLEVIRTVILPRRLLDRL

SEQ ID NO: 124 - protein, 18 aa, Artificial
  Artificial Peptide derived from HIV-1
YHRLRDLALIVTRIVELL

SEQ ID NO: 125 - protein, 18 aa, Artificial
  Artificial Peptide derived from HIV-1
RRGLLRVIRTVILALDIL

SEQ ID NO: 126 - protein, 21 aa, Artificial
  Artificial Peptide derived from HIV-1
RRGLLEVIRTVILLLDRLRHY

SEQ ID NO: 127 - protein, 21 aa, Artificial
  Artificial Peptide derived from HIV-1
RRGLLEVIRTVILALDRLRHY

SEQ ID NO: 128 - protein, 18 aa, Artificial
  Artificial Peptide derived from HIV-1
RRGLLEVIRTVILALDRL

SEQ ID NO: 129 - protein, 18 aa, Artificial
  Artificial Peptide derived from HIV-1
YHRLRDLLLIVCRIVELL

SEQ ID NO: 130 - protein, 21 aa, Artificial
  Artificial Peptide derived from HIV-1
YHRLRDLLLIVCRIVELLGRR

SEQ ID NO: 131 - protein, 21 aa, Artificial
  Artificial Peptide derived from HIV-1
YHRLRDLLLIVTRIVELLGRR

SEQ ID NO: 132 - protein, 18 aa, Artificial
  Artificial peptide derived from HIV-1
RRGLLEVIRCVILLLDRL

SEQ ID NO: 133 - protein, 18 aa, Artificial
  Artificial Peptide derived from HIV-1
RRGLLRVIRTVILLLDRL

SEQ ID NO: 134 - protein, 18 aa, Artificial
  Artificial Peptide derived from HIV-1
RRGLLEVIRTVILLLRRL

SEQ ID NO: 135 - protein, 21 aa, Artificial
  Artificial Peptide derived from HIV-1
RRGLLEVIRCVILLLDRLRHY

SEQ ID NO: 136 - protein, 21 aa, Artificial
  Artificial Peptide derived from HIV-1
RRGLLRVIRTVILLLDRLRHY

SEQ ID NO: 137 - protein, 21 aa, Artificial
  Artificial Peptide derived from HIV-1
RRGLLEVIRTVILLLRRLRHY

SEQ ID NO: 138 - protein, 20 aa, Artificial
  Artificial Peptide derived from HIV-1
YHKLKLLLIVTKIVELLGKK

SEQ ID NO: 139 - protein, 22 aa, Artificial
  Artificial Peptide derived from HIV-1
FLIRQLIRQLLTWQPILQYILQ

SEQ ID NO: 140 - protein, 25 aa, Artificial
  Artificial Peptide derived from HIV-1
FLIRQLIRLLTWLFSNCRTLLSEVY

SEQ ID NO: 141 - protein, 21 aa, Artificial
  Artificial Peptide derived from HIV-1
FLIRQLIRLLTWLFSNCRTLL

SEQ ID NO: 142 - protein, 21 aa, Artificial
  Artificial Peptide derived from HIV-1
LLTRCNSFLWTLLRILQRILF

SEQ ID NO: 143 - protein, 25 aa, Artificial
  Artificial Peptide derived from HIV-1
FLIRQLIRLLTWLFPNCRTLLSRVY

SEQ ID NO: 144 - protein, 25 aa, Artificial
  Artificial Peptide derived from HIV-1
YVRSLLTRCNSFLWTLLRILQRILF

SEQ ID NO: 145 - protein, 25 aa, Artificial
  Artificial Peptide derived from HIV-1
FLIKQLIKLLTWLFSNCKTLLSKVY

SEQ ID NO: 146 - protein, 23 aa, Artificial
  Artificial Peptide derived from HIV-1
RLVERIRQLTASRQLIPQLIQYV

SEQ ID NO: 147 - protein, 23 aa, Artificial
  Artificial Peptide derived from HIV-1
RLVRRIRQLTASRQLIPQLIQYV

SEQ ID NO: 148 - protein, 27 aa, Artificial
  Artificial Peptide derived from HIV-1
LLSRVYQILQPILQRLSATLQAIREVL

SEQ ID NO: 149 - protein, 28 aa, Artificial
  Artificial Peptide derived from HIV-1
LLSRVYQILQPILQRLCATLQRIREVLR

SEQ ID NO: 150 - protein, 28 aa, Artificial
  Artificial Peptide derived from HIV-1
RLVERIRQLTASLRQLIPQLIQYVRSLL

SEQ ID NO: 151 - protein, 28 aa, Artificial
  Artificial Peptide derived from HIV-1
LLSKVYQILQPILQKLSATLQKIKEVLK

SEQ ID NO: 152 - protein, 25 aa, Artificial
  Artificial Peptide derived from HIV-1
RLLTWLFSNCRTLLSRVYQILQPIL

SEQ ID NO: 153 - protein, 25 aa, Artificial
  Artificial Peptide derived from HIV-1
RLLTWLFSNRRTLLSRVYQILQEIL

SEQ ID NO: 154 - protein, 22 aa, Artificial
  Artificial peptide derived from HIV-1
RLLTWLRRTLLSRVYQILQEIL

SEQ ID NO: 155 - protein, 29 aa, Artificial
  Artificial Peptide derived from HIV-1
RIAGYGLRGLAVIIRCIIRGLNLIFEIIR

SEQ ID NO: 156 - protein, 29 aa, Artificial
  Artificial Peptide derived from HIV-1
RIAGYGLRGLAVIIRIICRGLNLIFEIIR

SEQ ID NO: 157 - protein, 30 aa, Artificial
  Artificial Peptide derived from HIV-1
RIAGYGLRGLAVIPRRICIRGLNLIFEIIR

SEQ ID NO: 158 - protein, 29 aa, Artificial
  Artificial Peptide derived from HIV-1
RIIEFILNLGRICIRIIVALGRLGYGAIR

SEQ ID NO: 159 - protein, 29 aa, Artificial
  Artificial Peptide derived from HIV-1
KIAGYGLKGLAVIIKICIKGLNLIFEIIK

SEQ ID NO: 160 - protein, 28 aa, Artificial
  Artificial Peptide derived from HIV-1
RVIRVVQGACRAIRHIPRRIRQGLRRIL

SEQ ID NO: 161 - protein, 28 aa, Artificial
  Artificial Peptide derived from HIV-1
RVIEVVQGACRAIEHIPRRIEQGLERIL

SEQ ID NO: 162 - protein, 28 aa, Artificial
  Artificial Peptide derived from HIV-1
RVIEVVQGACRAIEHIPRRIRQGLERIL

SEQ ID NO: 163 - protein, 28 aa, Artificial
  Artificial Peptide derived from HIV-1
RVIEVVQGACRAIRHIPRRIEQGLERIL

SEQ ID NO: 164 - protein, 28 aa, Artificial
  Artificial Peptide derived from HIV-1
RVIEVVQGACRASRHIPRRIRQGLERIL

SEQ ID NO: 165 - protein, 28 aa, Artificial
  Artificial Peptide derived from HIV-1
RVIEVVQGACRAIRHIPRRSRQGLERIL

SEQ ID NO: 166 - protein, 25 aa, Artificial
  Artificial Peptide derived from HIV-1
FLIRQLIELLTWLFSNCRTLLSEVY

SEQ ID NO: 167 - protein, 28 aa, Artificial
  Artificial Peptide derived from HIV-1
LLSEVYQILQPILQELSATLQRIREVLR

SEQ ID NO: 168 - protein, 21 aa, Artificial
  Artificial Peptide derived from HIV-1
YHELRDLLLIVTRIVELLGRE

SEQ ID NO: 169 - protein, 28 aa, Artificial
  Artificial Peptide derived from HIV-1
RVIEVVQGAYRAIRHIPRRIRQGLERIL

SEQ ID NO: 170 - protein, 28 aa, Artificial
  Artificial peptide derived from HIV-1
RVIRVVQRACRAIRHIVRRIRQGLRRIL

SEQ ID NO: 171 - protein, 31 aa, Artificial
  Artificial Peptide derived from HIV-1
RVIRVVQRACRAIRHIVRRIRQGLRRILRVV

SEQ ID NO: 172 - protein, 31 aa, Artificial
  Artificial Peptide derived from HIV-1
RWIRVVQRWCRAIRHIWRRIRQGLRRWLRVV

SEQ ID NO: 173 - protein, 12 aa, Artificial
  Artificial Peptide derived from HIV-1
RVVRVVRRVVRR

SEQ ID NO: 174 - protein, 24 aa, Artificial
  Artificial Peptide derived from HIV-1
RRVVRRVRRVVRRVVRVVRRVVRR

SEQ ID NO: 175 - protein, 36 aa, Artificial
  Artificial Peptide derived from HIV-1
VRRVVRRVVRVVRRVVRRVRRVVRRVVRVVRRVVRR

SEQ ID NO: 176 - protein, 42 aa, Artificial
  Artificial Peptide derived from HIV-1
RRVVRRVRRVVRRVVRVVRRVVRRVRRVVRRVVRVVRRVVRR

SEQ ID NO: 177 - protein, 48 aa, Artificial
  Artificial Peptide derived from HIV-1
RVVRVVRRVVRRVRRVVRRVVRVVRRVVRRVRRVVRRVVRVVRRVVRR

SEQ ID NO: 178 - protein, 12 aa, Artificial
  Artificial Peptide derived from HIV-1
RVVRVVRRWVRR

SEQ ID NO: 179 - protein, 24 aa, Artificial
  Artificial Peptide derived from HIV-1
RRWVRRVRRVWRRVVRVVRRWVRR

SEQ ID NO: 180 - protein, 36 aa, Artificial
  Artificial Peptide derived from HIV-1
VRRVWRRVVRVVRRWVRRVRRVWRRVVRVVRRWVRR

SEQ ID NO: 181 - protein, 48 aa, Artificial
  Artificial Peptide derived from HIV-1
RVVRVVRRWVRRVRRVWRRVVRVVRRWVRRVRRVWRRVVRVVRRWRVV

SEQ ID NO: 182 - protein, 6 aa, Artificial
  Artificial Peptide derived from HIV-1
RRWWRR

SEQ ID NO: 183 - protein, 12 aa, Artificial
  Artificial Peptide derived from HIV-1
RRWWRWWRRWWR

SEQ ID NO: 184 - protein, 18 aa, Artificial
  Artificial Peptide derived from HIV-1
WRRWWRRWWRWWRRWWRR

SEQ ID NO: 185 - protein, 24 aa, Artificial
  Artificial Peptide derived from HIV-1
RRWWRRWRRWWRRWWRWWRRWWRR

(19) United States
(12) Patent Application Publication
(10) Pub. No.: US 2009/0053278 A1
(43) Pub. Date: Feb. 26, 2009

(54) ANTI-MICROBIAL COMPOSITIONS AND DEVICES AND METHODS OF USING THE SAME

(76) Inventors: S. Robert Fatora, Monroeville, PA (US); Robert C. Dixon, Upper St. Clair, PA (US)

Correspondence Address: PEPPER HAMILTON LLP, ONE MELLON CENTER, 50TH FLOOR, 500 GRANT STREET, PITTSBURGH, PA, 15219, US

(21) Appl. No.: 11/923,643

(22) Filed: Oct. 24, 2007

Related U.S. Application Data
(60) Provisional application No. 60/856,395, filed on Nov. 3, 2006.
(60) Provisional application No. 60/859,451, filed on Nov. 17, 2006.

(51) Int. Cl. A61K 9/00; A61K 38/00; A61P 31/00

(52) U.S. Cl. 424/423; 514/12

(57) ABSTRACT

Embodiments of the present invention include medical devices that are coated with an antimicrobial peptide-based coating. Further embodiments include methods of coating the devices with the antimicrobial peptide-based coating, and treating a subject with the coated medical devices to prevent or relieve bacterial infections in the subject.

### CROSS-REFERENCE TO RELATED APPLICATIONS

This patent application claims priority to co-pending U.S. Provisional Patent Application No. 60/856,395, entitled “Fracture Fixation System Coated with Peptide-Based Antimicrobial”, filed on Nov. 3, 2006, the disclosure of which is incorporated herein by reference in its entirety. This patent application also claims priority to co-pending U.S. Provisional Patent Application No. 60/859,451, entitled “Tubes and Intubation Components Coated with a Peptide-Based Antimicrobial”, filed on Nov. 17, 2006, the disclosure of which is incorporated herein by reference in its entirety.

### GOVERNMENT INTERESTS

Not applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

1. Field of Invention

Not applicable

2. Description of Related Art

Not applicable

### BRIEF SUMMARY OF THE INVENTION

In one embodiment of the present invention, an implantable article or medical device comprising an implant for a mammal having a biologically compatible surface is provided. In certain embodiments, a portion of the surface may further include a therapeutically effective amount of an antimicrobial peptide. The antimicrobial peptide may be immobilized or temporarily attached to the surface of the implant or device. Preferably, the antimicrobial peptides are alpha helical. More preferably, the antimicrobial peptides are selected from lentivirus lytic peptides (LLPs), lytic base unit peptides (LBUs) and engineered cationic antimicrobial peptides (eCAPs).

In an embodiment, a method for immobilizing antimicrobial peptides on an implantable article or medical device, may include providing a medical device, and applying a linking layer to at least a portion of a surface of the medical device, followed by reacting an antimicrobial peptide to the linking layer.

In another embodiment, a fracture fixation system may include at least one intramedullary nail. The nail may be covered with a substantially uniform layer of peptide based antimicrobial. The fracture fixation system may include at least one cannulated bone screw or bolt, which contains a plurality of apertures at a bore end, and where at least one cannulated bone screw or bolt is covered with the substantially uniform layer of peptide based antimicrobial. In embodiments, a composite material may include a peptide-based antimicrobial with a bone growth promoter perfused through the plurality of apertures at the bore end.

In yet another embodiment, an intubation system may include at least one tube. The one tube may include an internal surface and an external surface that are coated substantially uniformly with an immobilized antimicrobial peptide. At least one cuff may be included, where the at least one cuff has an internal surface and an external surface that are coated substantially uniformly with an immobilized antimicrobial peptide. At least one tip may be included, where the tip has an internal surface and an external surface that are coated substantially uniformly with an immobilized antimicrobial peptide.

In still yet another embodiment, a method of preventing infection in a subject comprising coating at least a portion of a surface of a medical device with a therapeutically effective amount of an antimicrobial peptide, and implanting the coated medical device in the subject is provided. The antimicrobial peptide may be immobilized or semi-immobilized on the surface of the implant. Preferably, the antimicrobial peptides are alpha helical. More preferably, the antimicrobial peptides are selected from LLPs, LBUs and eCAPs.

### DESCRIPTION OF DRAWINGS

For a fuller understanding of the nature and advantages of the present invention, reference should be had to the following detailed description taken in connection with the accompanying drawings, in which:

FIG. 1 depicts an antibacterial peptide covalently bonded to a titanium medical device through an organosilane linking group.

FIG. 2 is an elevation and side view of an embodiment of an intramedullary (IM) nail for setting a long leg or arm bone fracture.

FIG. 3 is an elevation and side view of an embodiment of an IM nail covered with a uniform layer of a peptide-based antimicrobial for setting a leg or arm fracture.

FIG. 4 is a cross sectional view of an embodiment of a cannulated bone screw (FIG. 4A) or bolt (FIG. 4B) with a plurality of apertures at its bore end for use in anchoring the IM nail to the human leg and arm bones.

FIG. 5 is cross sectional view of an embodiment of a cannulated bone screw (FIG. 5A) or bolt (FIG. 5B) with a plurality of apertures at its bore end and covered with a uniform layer of a peptide-based antimicrobial for use in anchoring the IM nail to the human leg and arm bones.

FIG. 6 is an elevation view of an embodiment of a cannulated bone screw (FIG. 6A) or bolt (FIG. 6B) with a plurality of apertures at its bore end which allows for a peptide-based antimicrobial or a composite material, comprised of a peptide-based antimicrobial with a bone growth promoter, to perfuse through the plurality of apertures at the bore end and is covered with a uniform layer of a peptide-based antimicrobial for use in anchoring the IM nail to the human leg and arm bones.

FIG. 7 is a schematic view of the fracture fixation system (femur shown only) of embodiments herein.

FIG. 8 is a cross section view of an embodiment of a tube that may be used within an endotracheal apparatus for intubating a patient's airway, catheter or shunt.

FIG. 9 is a cross section view of an embodiment of a tube covered internally and externally with a uniform layer of a peptide-based antimicrobial that may be utilized within an endotracheal apparatus for intubating a patient's airway, catheter or shunt.

FIG. 10 is a schematic view of an embodiment of an inflatable cuff covered internally and externally with a uniform layer of a peptide-based antimicrobial that may be used within an endotracheal apparatus for intubating a patient's airway.

FIG. 11 is a cross section view of an embodiment of a distal tip covered internally and externally with a uniform layer of a peptide-based antimicrobial that could be used within an endotracheal apparatus for intubating a patient's airway, catheter or shunt.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “nail” is a reference to one or more nails and equivalents thereof known to those skilled in the art, and so forth.

As used herein, the term “about” means plus or minus 10% of the numerical value of the number with which it is being used. Therefore, about 50% means in the range of 45%-55%.

“Administering” when used in conjunction with a therapeutic means to administer a therapeutic directly into or onto a target tissue or to administer a therapeutic to a patient whereby the therapeutic positively impacts the tissue to which it is targeted. Thus, as used herein, the term “administering”, when used in conjunction with antimicrobial peptides, can include, but is not limited to, providing an antimicrobial peptide into or onto the target tissue from a biomedical device that is coated with an antimicrobial peptide.

As used herein, the term “antimicrobial” refers to the ability of the peptides of the invention to prevent, inhibit or destroy the growth of microbes such as bacteria, fungi, protozoa and viruses.

The terms “biomedical device”, “medical device”, “implantable article” or “implantable medical device” as used herein includes an instrument, apparatus, implement, machine, contrivance, implant, or other similar or related article, including a component part, or accessory which is intended for use in the diagnosis of disease or other conditions, or in the cure, mitigation, treatment, or prevention of disease, in man or other animals, or intended to affect the structure or any function of the body of man or other animals. This includes any device that is used in the human body, either permanently or for a therapeutic time, including devices such as, but not limited to, fluid infusion devices, shielded needle products, catheters, cannulas, endotracheal tubes, indwelling catheters, implants, shunts, stents, intubation systems, cardiac assist devices, neurosurgical-ventricular shunts, fracture fixations systems, mechanical heart valves, vascular grafts, pacemakers, bladder catheters, central venous catheters, penile implants and mammary implants and any other indwelling medical device.

The term “improves” is used to convey that the present invention changes either the appearance, form, characteristics and/or the physical attributes of the tissue or bodily fluid to which it is being provided, applied or administered. The change in form may be demonstrated by any of the following alone or in combination: reduction of nosocomial infections, such as for example, but not limited to, ventilator-associated pneumonia (VAP), blood stream infections, and urinary tract infections.

The term “inhibiting” includes the administration of a compound of the present invention to prevent the onset of the symptoms, alleviating the symptoms, or eliminating the disease, condition or disorder.

As used herein, the term “peptide” refers to an oligomer of at least two contiguous amino acids, linked together by a peptide bond.

By “pharmaceutically acceptable”, it is meant the carrier, diluent or excipient must be compatible with the other ingredients of the formulation and not deleterious to the recipient thereof.

As used herein, the term “therapeutic” means an agent utilized to treat, combat, ameliorate, prevent or improve an unwanted condition or disease of a patient. In part, embodiments of the present invention are directed to the prevention of infections associated with biomedical devices installed in a subject permanently or for a therapeutic time.

A “therapeutic time” as used herein, is a time required to effectively treat, combat, ameliorate, prevent or improve an unwanted condition or disease of a subject.

A “therapeutically effective amount” or “effective amount” of an antibacterial peptide is a predetermined amount calculated to achieve the desired effect, i.e., to inhibit, block, or reverse the infection of bodily tissue and/or fluids when in the presence of a biomedical device. In embodiments, the effective amount of antibacterial peptide is the amount coated on a biomedical device that prevents the device from being coated with a “biofilm”. A biofilm is an excellent growth medium for bacteria and ultimately precipitates infection. In other embodiments, the effective amount of an antibacterial peptide is the amount coated on a biomedical device that prevents adhesion of bacteria, subsequent colonization and maturation of the bacteria, and the formation of a biofilm. The specific dose of an antimicrobial peptide in a coating on a biomedical device that is administered according to this invention to obtain therapeutic and/or prophylactic effects will, of course, be determined by the particular circumstances surrounding the case, including, for example, the peptide administered, the nature of the biomedical device, and the contemplated infection to be blocked or treated. A therapeutically effective amount of antimicrobial peptide of this invention is typically an amount such that when it is administered as a component of a biomedical device or a coating on a biomedical device, it is sufficient to achieve an effective inhibition of a biofilm on a biomedical device.

The terms “treat,” “treated,” or “treating” as used herein refers to both therapeutic treatment and prophylactic or preventative measures, wherein the object is to prevent or slow down (lessen) a bacterial infection, or to obtain beneficial or desired clinical results, such as, but not limited to osteogenesis in a fracture fixation system involving intramedullary nailing of diaphyseal fractures. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

Generally speaking, the term “tissue” refers to any aggregation of similarly specialized cells which are united in the performance of a particular function.

Embodiments of the present invention are directed to medical devices coated with antimicrobial peptides. In preferred embodiments, the medical devices are permanently or semi-permanently introduced into the body of a subject, preferably a human. In preferred embodiments, the medical devices are selected from fluid infusion devices, shielded needle products, catheters, cannulas, endotracheal tubes, indwelling catheters, implants, shunts, stents, intubation systems, cardiac assist devices, neurosurgical-ventricular shunts, fracture fixations systems, mechanical heart valves, vascular grafts, pacemakers, bladder catheters, central venous catheters, penile implants and mammary implants and any other indwelling medical devices, more preferably, fracture fixation systems, tubes that penetrate bodily tissue of a patient, intubation system components. Preferably, the antimicrobial peptides comprise peptides selected from linear lytic peptides (LLPs), lytic base unit peptides (LBUs), and engineered cationic peptides (eCAPs). The antimicrobial peptide may be immobilized or temporarily attached to the surface of the implant or device.

Further embodiments of the present invention are directed to methods of preventing or treating an infection comprising implanting a medical device coated with an antimicrobial peptide into a subject.

Further embodiments of the present invention are directed to coating antimicrobial peptides on medical devices. The antimicrobial peptide may be immobilized or temporarily attached to the surface of the device.

Antimicrobial peptides that are useful in embodiments of the present invention include those derived from selected amino acid sequences in viral transmembrane proteins. In particular, the peptides may be derived from lentiviruses, primarily human immunodeficiency virus (HIV), simian immunodeficiency virus (SIV), and equine infectious anemia virus (EIAV). These are lytic peptides derived from lentiviruses and are designated by the term “LLPs”, as disclosed in U.S. Pat. No. 5,714,577, which is incorporated herein in its entirety. In contrast to other antimicrobial peptides, which are specifically encoded by their own genes, LLPs are unique in that they are derived from naturally occurring sequences that are part of a larger folded protein.

In another embodiment of the invention, the antimicrobial peptides are structural and functional analogs of the naturally occurring parent peptides which exhibit selective toxicity for microorganisms.

As used herein, the term “analog” refers to a peptide which contains substitutions, rearrangements, deletions, additions and/or chemical modifications in the amino acid sequence of parent peptide, and retains the structural and functional properties of the parent peptide.

In another embodiment of the invention, the antimicrobial peptides are structural and functional homologs of the naturally occurring parent peptides which exhibit selective toxicity for microorganisms.

As used herein, the term “homolog” refers to a peptide, the sequence of which is at least 80% homologous to the amino acid sequence of a parent peptide, and retains the structural and functional properties of the parent peptide.

In embodiments of the present invention, the amino acid sequences of the antimicrobial peptides of the invention correspond to or are analogous to or homologous to peptides LLP1 and LLP2, which, in turn, correspond to residues 828-855 and 768-788 of the HIV-1 TM protein (gp41) (strain HXB2R), respectively; peptides SLP-1, SLP-2A and SLP-2B (SLP2 region), which, in turn, correspond to residues 852-879, 771-795 and 790-817 of the SIV TM protein (MM239 strain of SIV), respectively; and peptide ELP, which corresponds to residues 808-836 of EIAV (Wyoming strain). The striking feature of these sequences is their lack of sequence homology to known cytolytic peptides (e.g. magainins); however, each is rich in positively charged residues and is predicted to form an amphipathic helix. This structure imparts to each of the peptides a unique but potent antimicrobial activity.

The antimicrobial peptides of the invention are unique in their functional properties. In general, cytolytic peptides can be classified into two major functional types. Antibacterial peptides (magainins and cecropins, for example) specifically kill bacteria. Hemolytic peptides, on the other hand, generally both kill bacteria and lyse red blood cells; melittin from bee venom is an example of such a peptide. The antimicrobial peptides useful in the present invention are moderately hemolytic; they do lyse red blood cells, but only at high concentrations. The unique structure of the antimicrobial peptides of the invention imparts high potency while maintaining selectivity. The structural properties defining the antimicrobial peptides useful in embodiments of the present invention include, inter alia, a significant number of positively charged amino acid residues and the ability to form three-dimensional amphipathic helical structures. Functional properties include, inter alia, a selective antimicrobial cytolytic activity, but minimal cytolytic activity toward mammalian cells.

The structural formulae of the exemplary antimicrobial peptides useful in the invention corresponding to regions of TM proteins derived from HIV-1, SIV, and EIAV are listed in TABLE 1.

TABLE 1
Parent Antimicrobial Peptides

| | | |
|---|---|---|
| Peptide Name | Amino Acid Sequence | Sequence Source¹ |

| | | | |
|---|---|---|---|
| LLP1 | (SEQ ID NO. 1) | RVIEVVQGACRAIRHIPRRIRQGLERIL | HIVHXB2R 828-855 |
| LLP2 | (SEQ ID NO. 2) | YHRLRDLLLIVTRIVELLGRR | HIVHXB2R 768-788 |
| SLP1 | (SEQ ID NO. 3) | DLWETLRRGGRWILAIPRRIRQGLELTL | SIVMM239 852-879 |
| SLP2A | (SEQ ID NO. 4) | FLIRQLIRLLTWLFSNCRTLLSRVY | SIVMM239 771-795 |
| SLP2B | (SEQ ID NO. 5) | LLSRVYQILQPILQRLSATLQRIREVLR | SIVMM239 790-817 |
| ELP | (SEQ ID NO. 6) | RIAGYGLRGLAVIIRICIRGLNLIFEIIR | EIAV 808-836 |

wherein: A=Ala=Alanine R=Arg=Arginine N=Asn=Asparagine D=Asp=Aspartic acid B=Asx=Asparagine or aspartic acid C=Cys=Cysteine Q=Gln=Glutamine E=Glu=Glutamic acid Z=Glx=Glutamine or glutamic acid G=Gly=Glycine H=His=Histidine I=Ile=Isoleucine L=Leu=Leucine K=Lys=Lysine M=Met=Methionine F=Phe=Phenylalanine P=Pro=Proline S=Ser=Serine T=Thr=Threonine W=Trp=Tryptophan Y=Tyr=Tyrosine V=Val=Valine. Sequences of the family of LLPs derived from HIV and SIV envelope proteins are consistent with the numbering in Myers. The sequence of ELP, the peptide derived from the ENV protein of EIAV, is from the Wyoming strain (Rushlow et al., 1986).

A peptide analog or homolog within the scope of the present invention may be identified by the following criteria: (1) the parent peptide of the analog is an antimicrobial peptide having a sequence which corresponds to a vital TM protein, particularly a lentivirus TM protein; (2) the amino acid sequence of the peptide is capable of forming an amphipathic helix and contains a number of positively charged residues; (3) the peptide is selectively antimicrobial in its biological function and has minimal cytolytic activity toward mammalian cells.

In the design of the peptide analogs of the antimicrobial peptides useful in embodiments of the present invention, the allowed amino acid interchanges which are contemplated include, inter alia, the substitution of an individual residue in the peptide with a residue that falls within the same chemical subset, e.g., a hydrophobic amino acid replaced by the same or a positively charged residue with the same. This degree of substitution allows for the construction of peptide analogs from the parent structure which retain the structural and functional properties of the parent peptide, without undue experimentation.

Analogs may also contain non-conservative amino acid interchanges provided that structural and functional properties are retained or enhanced. A singular characteristic of the antimicrobial peptides useful in embodiments of the present invention is the presence of a significant number of positively charged residues, especially arginine. Analogs and homologs include those that encompass substitutions which retain the overall charge characteristics of the peptides. Preferably, the peptides useful in embodiments of the present invention have a net charge of at least +3 at neutral pH. Net charge is calculated by adding the sum of the charge value of positively charged amino acids (arginine, lysine, histidine) (+1) and the charge value of the negatively charged amino acids (aspartic acid, glutamic acid) (−1). Thus, the positively-charged arginines in the peptides may be substituted by histidine or lysine so as to retain positively charged residues. Analogs may be designed which increase the number of positively charged amino acids so long as the antimicrobial activity of the peptide is not diminished, for example, the number of arginine residues may be increased. An analog which increased the number of positive charges in an LLP1 analog peptide has been shown to be more toxic to bacteria than the parent LLP1.

Additional analogs of the peptides useful in embodiments of the present invention can have an altered number of hydrophobic amino acids based on the parent peptide, producing peptides having altered specificity. For example, an increase in hydrophilic residues appears to reduce antimicrobial effectiveness. However, such changes appear to increase antimicrobial specificity by reducing undesired hemolytic activity. Therefore, based on the teachings and guidance herein, one skilled in the art can design analogs useful in embodiments of the invention which have a desired potency and selectivity.

In the design of peptide homologs of the antimicrobial peptides useful in embodiments of the invention, the amino acid changes which are contemplated include, inter alia, the replacement of amino acid residues in a parent peptide such that the homologous peptide retains the structural and functional properties of the parent peptide.

A primary common and recognizable feature of the antimicrobial peptides is their secondary structure, or more specifically, their potential to form amphipathic structures, which may be in the form of an alpha-helix or a beta conformation. An alpha-helix motif, for example, comprises residues arranged such that 3.5 amino acid residues complete 1 turn of the helix. An estimate of amphipathicity may therefore be made by examination of the amino acid sequence; for example, peptides comprising amino acid residues arranged in a hydrophobic-hydrophobic-hydrophilic-hydrophilic repeating motif are highly likely to form an alpha-helix. Amino acid residues arranged to alternate in a hydrophobic-hydrophilic-hydrophobic-hydrophilic repeating motif are likely to form a beta conformation. Such “ideal” motifs are found in the antimicrobial peptides of the invention and as such may be used by those skilled in the art as a foundation for engineering additional amphipathic peptide analogs of the invention without great difficulty based on the teachings herein. The antimicrobial peptides useful in embodiments of the invention may further contain proline or glycine, amino acid residues which can be tolerated within a general amphipathic structure and may indicate demarcations between different amphipathic regions. These residues may impart a structure which enhances the activity and selectivity of a peptide because of a bend or kink between helices. For example, a solidly helical structure may be less selective (e.g. LLP2, SLP2A). Homologs may also be engineered, using these structural considerations that are at least 80% homologous to the amino acid sequence of a parent peptide, and retain the structural and essential antimicrobial functional properties of the parent peptide.

Analogs and homologs in which the amphipathicity of a peptide is increased by additions, deletions and/or substitutions of amino acids in a parent peptide are within the scope of the invention.

Analogs and/or homologs of the invention preferably contain at least one cysteine which, by virtue of its capacity to form a disulfide bond, can confer high potency and a very high degree of bactericidal activity to a peptide containing such a residue. A peptide preferably contains a single cysteine residue to ensure that any disulfide bond formed by the cysteine would be intermolecular and result in a disulfide-inked dimeric peptide (e.g. bis-LLP1). The residue to be replaced by cysteine is preferably neither very hydrophobic nor basic and lies on the interface of the hydrophilic and hydrophobic faces of the amphipathic structure when modeled as such. Computer modeling programs such as “Helicalwheel” may be used to design such peptides.

The antimicrobial peptides of the invention generally comprise a positively charged C-terminus. However, those peptides having this characteristic generally have some hemolytic activity, and analogs which optimize antimicrobial selectivity (i.e., decrease hemolytic activity) may be those which replace the positively charged C-terminus with negatively charged or hydrophobic residues. Since reduction of the basic character of the C-terminus may provide antimicrobial selectivity, analogs are provided in which the amino acids of the C-terminus region may be reversed in situ, or, alternatively, the N-terminus and C-terminus regions may be interchanged. The peptide is then comprised of a positively charged N-terminus and a hydrophobic C-terminus.

Analogs and homologs which are chimeras of particular antimicrobial peptides and/or other cytolytic peptides are within the scope of the present invention, provided that the structural and functional properties described herein are retained.

In another embodiment of the invention, the use of D-amino acids in place of L-amino acids in the peptides may provide increased metabolic stability, since peptides containing D-amino acids are resistant to mammalian proteases, which generally cleave peptides composed of L-amino acids

Embodiments of the present invention may also include the use of peptide analogs and homologs which are truncated, i.e., shorter than the parent amino acid sequence or to truncated parent peptide fragments. A minimal length required to effectuate ion-channel formation in membranes is believed to be a peptide of 8-12 amino acid residues in length. It has been suggested that the antimicrobial peptides may dimerize so as to comprise the approximately 20 amino acid length believed to be required to transverse a membrane. As discussed above, the inclusion of a cysteine residue in an antimicrobial peptide is of importance in facilitating the formation of intramolecular or intermolecular disulfide bonds which can stabilize a dimeric peptide. A 21-amino acid segment of LLP1 was capable of pore formation in planar lipid bilayers in vitro, although it was not tested for antimicrobial activity. The design of analogs of minimal length can optimize potency of the peptides in terms of effectiveness per mass.

The following analogs and homologs, derived from the parent peptides shown in TABLE 1, are also exemplary of the antimicrobial peptides useful in embodiments of the present invention, and have the following primary structural formulae:

LLP1 Analogs:

SEQ ID NO. 7 and SEQ ID NO. 10 through SEQ ID NO. 72

SLP-1 Analogs:

SEQ ID NO. 73 through SEQ ID NO. 107

LLP2 Analogs:

SEQ ID NO. 108 through SEQ ID NO. 138

SLP2A Analogs:

SEQ ID NO. 139 through SEQ ID NO. 145

SLP2B Analogs:

SEQ ID NO. 146 through SEQ ID NO. 151

SLP2 Region Analogs:

SEQ ID NO. 152 through SEQ ID NO. 154

ELP Analogs:

SEQ ID NO. 155 through SEQ ID NO. 159

In one preferred embodiment, the antimicrobial peptides useful in the present invention have the following structural formula:

SEQ ID NO. 4 through SEQ ID NO. 5

SEQ ID NO. 7 through SEQ ID NO. 9

Analogs and homologs of other naturally occurring lytic peptides derived from other lentivirus proteins are also within the scope of the invention. Peptides may be derived from proteins of any lentivirus or any DNA or RNA virus including, but not limited to, HIV-1, HIV-2, SIV, EIAV, feline immunodeficiency virus (FIV), bovine immunodeficiency virus (BIV), visna virus and all clades, subclasses and isolates thereof.

In another embodiment of the invention, antimicrobial peptides which are derived from, and are analogs of, the LLP1 peptide parent sequence corresponding to amino acids 828-856 of the HIV-1 viral isolate HXB2R Env and include SA-5 (SEQ ID NO. 1), LSA-5 (SEQ ID NO. 2) and WLSA-5 (SEQ ID NO. 3) (see Table 1 below) are useful to coat medical devices. The antimicrobial activity of other LLP1 peptide analogues has been previously described (see Tencza et al., 1999, Journal of Antimicrobial Chemotherapy 44:33-41, U.S. Pat. No. 5,714,577 of Montelaro et al. and U.S. Pat. No. 5,945,507 of Montelaro et al.). Antimicrobial peptides useful in this invention include those disclosed in U.S. Pat. No. 6,887,847, which is incorporated herein in its entirety. In embodiments of the invention, the antimicrobial peptides are LLP1 analogs having modifications based on the following principles: (i) optimizing amphipathicity, (ii) substituting arginine (Arg) on the charged face and/or valine (Val) or tryptophan (Trp) on the hydrophobic face with another amino acid, and (iii) increasing peptide length (referred to collectively herein as Lytic Base Unit peptides (LBU peptides), e.g. LBU-2, SEQ ID NO. 174; LBU-3, SEQ ID NO. 175; LBU-3.5, SEQ ID NO. 176; LBU-4, SEQ ID NO. 177; WLBU-1, SEQ ID NO. 178, WLBU-2, SEQ ID NO. 179, WLBU-3, SEQ ID NO. 180; and WLBU-4, SEQ ID NO. 181; see Table 2). The LBU peptides deviate greatly from the parent LLP1, for example, LBU-2 and LBU-3 deviate from the parent LLP1 sequence by greater than 90%.

TABLE 2

| | | |
|---|---|---|
| SA-5 | RVIRV VQRAC RAIRH IVRRI | (SEQ ID NO. 170) |
| | RQGLR RIL | |
| LSA-5 | RVIRV VQRAC RAIRH IVRRI | (SEQ ID NO. 171) |
| | RQGLR RILRV V | |
| WLSA-5 | RWIRV VQRWC RAIRH IWRRI | (SEQ ID NO. 172) |
| | RQGLR RWLRV V | |
| LBU-1 | RVVRV VRRVV RR | (SEQ ID NO. 173) |
| LBU-2 | RRVVR RVRRV VRRVV RVVRR | (SEQ ID NO. 174) |
| | VVRR | |
| LBU-3 | VRRVV RRVVR VVRRV VRRVR | (SEQ ID NO. 175) |
| | RVVRR VVRVV RRVVRR | |
| LBU-3.5 | RRVVR RVRRV VRRVV RVVRR | (SEQ ID NO. 176) |
| | VVRRV RRVVR RVVRV VRRVV | |
| | RR | |
| LBU-4 | RVVRV VRRVV RRVRR VVRRV | (SEQ ID NO. 177) |
| | VRVVR RVVRR VRRVV RRVVR | |
| | VVRRV VRR | |
| WLBU-1 | RVVRV VRRWV RR | (SEQ ID NO. 178) |
| WLBU-2 | RRWVR RVRRV WRRVV RVVRR | (SEQ ID NO. 179) |
| | WVRR | |
| WLBU-3 | VRRVW RRVVR VVRRW VRRVR | (SEQ ID NO. 180) |
| | RVWRR VVRVV RRWVR R | |
| WLBU-4 | RVVRV VRRWV RRVRR VWRRV | (SEQ ID NO. 181) |
| | VRVVR RWVRR VRRVW RRVVR | |
| | VVRRW RVV | |

The LLP1 analogue peptides and the LBU peptides (collectively referred to herein as “engineered LLPs” (eLLPs)) useful in embodiments of the present invention have a broader spectrum of activity (i.e., the ability to kill highly resistant bacteria) and increased potency (i.e., lowering the molar concentration required to kill bacteria) when compared with previously described LLP1 analogs. The eLLPs of the present invention are highly inhibitory to microorganisms under physiologic salt concentrations, function in the presence of synovial fluid, and demonstrate only minimal toxicity in animal models. As a result, the eLLPs may be defined as selective antimicrobial agents. In addition, the peptides useful in embodiments of the present invention function by disrupting bacterial membranes and are active when bound to a solid phase. The ability of these peptides to maintain activity when bound to a solid phase is a significant advantage over conventional antibiotics.

The antimicrobial peptides useful in embodiments of the present invention, collectively referred to herein as “eLLPs”, exhibit antimicrobial activity against diverse microorganisms, and are analogs of the LLP1 peptide corresponding to amino acids 828-856 of the HIV-1 viral isolate HXB2R Env TM. The eLLPs comprise Arg-rich sequences, which, when modeled for secondary structure, display high amphipathicity and hydrophobic moment. The eLLPs are highly inhibitory to microorganisms, but significantly less toxic to mammalian cells. As a result, these peptides can be characterized as selective antimicrobial agents. In addition, the eLLPs of the present invention include LLP1 peptide analogs comprising modifications based on the following principles: (i) optimizing amphipathicity, (ii) substituting Arg on the charged face and/or Val or Trp on the hydrophobic face, and (iii) increasing peptide length, collectively referred to herein as LBU peptides.

In another embodiment of the invention, engineered cationic antimicrobial peptides (eCAP) may be useful in embodiments of the present invention. These arginine/tryptophan-rich peptides are presented in Table 3 below. Several of these eCAP peptides showed significant activity in reducing HIV-1 infectivity of cells.

TABLE 3

| | | |
|---|---|---|
| Peptide | Sequence | Comments |

| | | |
|---|---|---|
| WR6 | RRWWRR | SEQ ID NO. 182 |
| WR12 | RRWWRWWRRWWR | SEQ ID NO. 183 |
| WR18 | WRRWWRRWWRWWRRWWRR | SEQ ID NO. 184 |
| WR24 | RRWWRRWRRWWRRWWRWWRRWWRR | SEQ ID NO. 185 |

Accordingly, one embodiment of the present invention provides a medical device comprising a medical device coated with an antimicrobial peptide. In a preferred embodiment, the antimicrobial peptide is selected from LLPs, LBUs and eCAPs. Preferably, the antimicrobial peptide is selected from SEQ ID NO. 1 through SEQ ID NO. 185. The antimicrobial peptide may be immobilized or temporarily attached to the surface of the implant or device.

In a preferred embodiment, the antimicrobial peptides are present on the medical device in a therapeutically effective amount. The medical device may be any device that is implanted into or onto the body, preferably, for example, a fracture fixation system, intubation systems, endotracheal tubes, nasogastric tubes, catheters, and shunts.

In certain embodiments the antimicrobial peptide is coated on the medical device such that the antimicrobial peptides substantially cover the entire surface of the device. In certain embodiments, the antimicrobial peptide is coated on the medical device such that the antimicrobial peptide covers a partial surface or portion of the surface of the device, such as, for example, the portion of the device that it inserted into or placed on the body of the subject. The antimicrobial peptide may be immobilized or temporarily attached to the surface of the implant or device. In further embodiments, the antimicrobial peptide is coated on the medical device and further includes a second therapeutic agent.

In one preferred embodiment, the antimicrobial peptides useful in the present invention have the following structural formula: SEQ ID NO. 4, SEQ ID NO. 5, SEQ ID NO. 8, SEQ ID NO. 9, SEQ ID NO. 173 through SEQ ID NO. 185.

Another embodiment of the instant invention includes a fracture fixation system that is coated with a peptide based antimicrobial. Preferably, the antimicrobial peptide is an alpha helical peptide. More preferably, the antimicrobial peptide is selected LLPs, LBUs and eCAPs. More preferably, the antimicrobial peptide is selected from SEQ ID NO. 1 through SEQ ID NO. 185. Exemplary fracture fixation systems include an IM nail, screw and bolt or similar apparatus.

Preferred antimicrobial peptides to be used as antimicrobial coatings on components of a fracture fixation system, include but are not limited to, LLPs, LBUs and eCAPs, as presented above, including preferably, SEQ ID NO. 4, SEQ ID NO. 5, SEQ ID NO. 8, SEQ ID NO. 9, SEQ ID NO. 173 through SEQ ID NO. 185.

Embodiments herein include a uniform antimicrobial coating, as disclosed hereinabove, for a fracture fixation system. In these embodiments, the antimicrobial coating substantially covers the entire surface of the fracture fixation system. Still other embodiments include a partial antimicrobial coating for a fracture fixation system. In these embodiments, the coating may only cover a portion of the surface of the medical device. The portion of the device that is coated may include only a portion that is inserted into a patient's body. In still another embodiment, an antimicrobial coating on a fracture fixation system may also include a second agent, such as, but not limited to, a bone growth promoter. A bone growth promoter may include any agent known now or hereinafter to one of ordinary skill in the art. Still further embodiments of the present invention include a fracture fixation that is coated with a therapeutically effective amount of antimicrobial peptide. The antimicrobial peptide may be immobilized or temporarily attached to the surface of the system or portions thereof. Another embodiment of the present invention includes methods to coat a fracture fixation system with antimicrobial peptides. Preferred methods for coating a fracture fixation system with antimicrobial peptides are the same as those for coating a medical device and are disclosed hereinabove.

Embodiments of the invention pertain to a fracture fixation system coated with a peptide-based antimicrobial and permits deposition of a novel composite of peptide-based antimicrobial. An embodiment of the present invention includes: a) an IM nail covered with a uniform layer of a peptide-based antimicrobial; b) a cannulated bone screw or bolt with a plurality of apertures at its bore end which allows for the perfuse of a peptide-based antimicrobial or a novel composite material and is covered with a uniform layer of a peptide-based antimicrobial; and c) a novel composite material, comprised of a peptide-based antimicrobial with a bone growth promoter, to perfuse through the plurality of apertures at the bore end to inhibit biofilm formation and/or kills the bacteria responsible for infection at or near the bore end apertures.

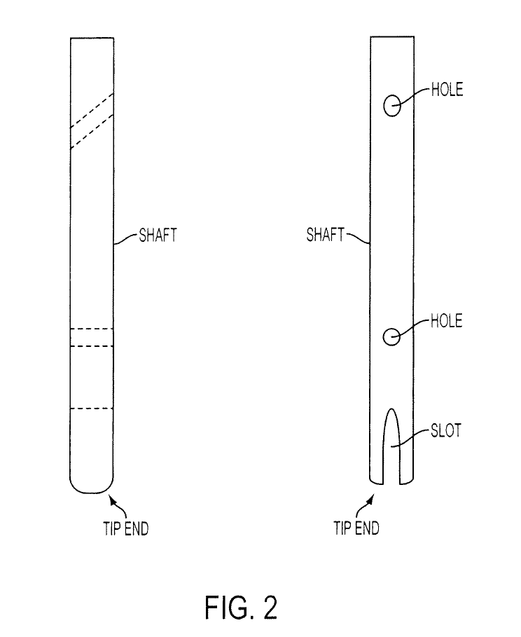

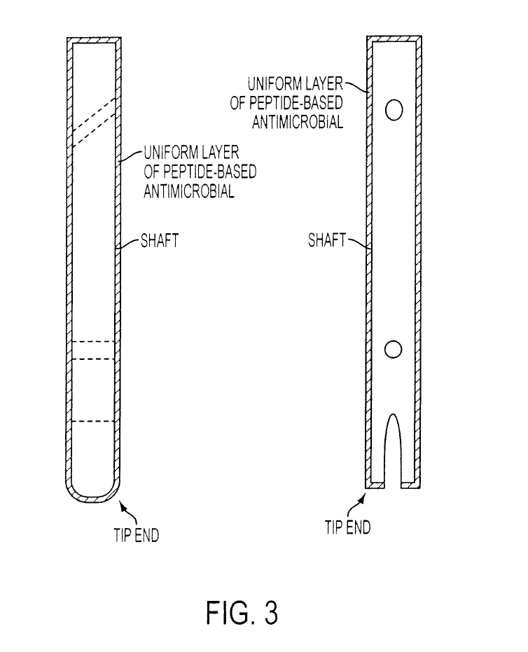

For example, the IM nail may include a hollow circular cross-section shaft having a head and tip end as shown in FIG. 2. It can be manufactured from plastic, nickel-titanium alloy, or stainless steel or other similar suitable materials. The IM nail may have two (2) holes and a slot at the tip end through which securing bone screws or bolts can be inserted transverse into the IM nail and leg or arm bone to secure the IM nail in the desired position. The IM nail slot may be slightly larger than the bone screw or bolt's diameter allowing for the engagement of the IM nail by the bone screw or bolt previously inserted through the leg or arm bone medulla. The IM nail of FIG. 2 may be uniformly covered with a uniform layer of peptide-based antimicrobial as shown in FIG. 3.

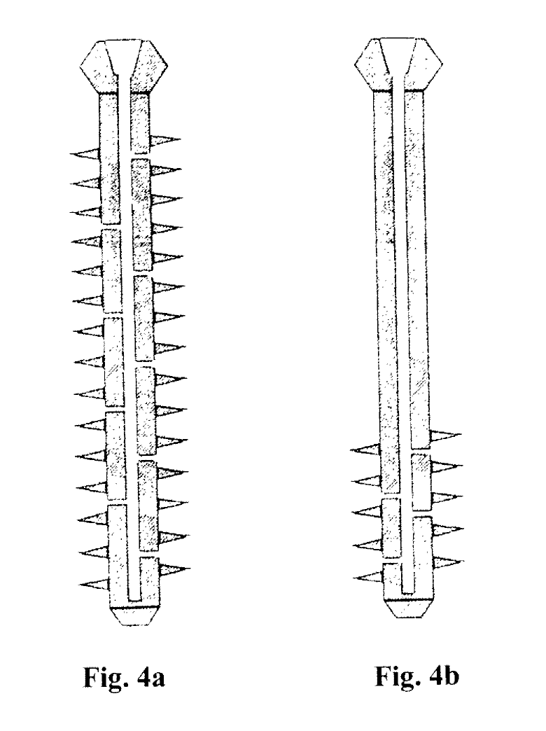

In another embodiment, a bone screw may have a head with an opening formed therein, a closed end bore with a plurality of apertures for a tip, and a cannulated, circular cross-section shaft with a thread segment along nearly its entire length as shown in FIG. 4A.

In another embodiment, a bone bolt may have a head with an opening formed therein, a closed end bore with a plurality of apertures for a tip, and a cannulated, circular cross-section shaft with a thread segment along a certain portion of its entire length as shown in FIG. 4B.

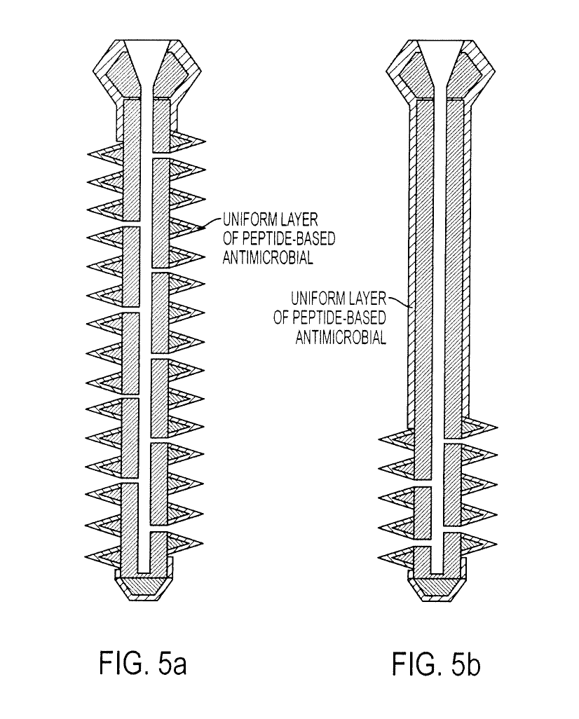

In another embodiment, the bone screw and bolt of FIGS. 4A and 4B may be covered by a uniform layer of peptide-based antimicrobial as shown in FIG. 5A and FIG. 5B, respectively.

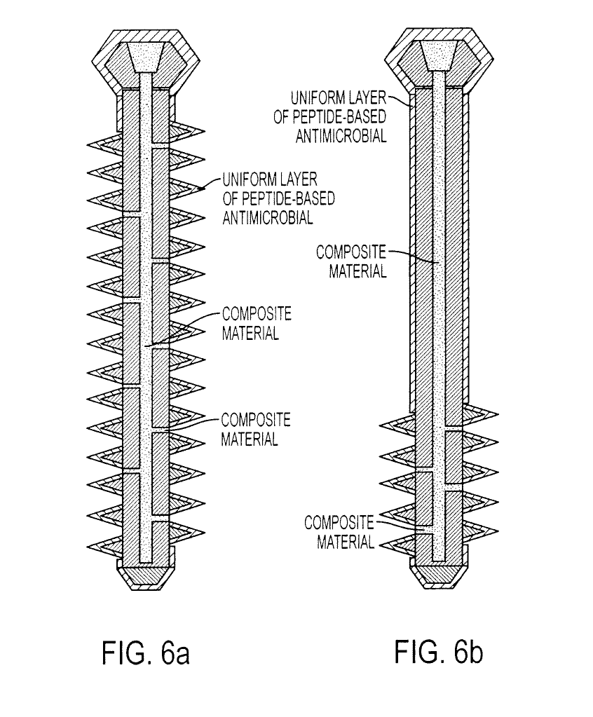

In a further embodiment, a peptide-based antimicrobial or a composite material comprised of a peptide-based antimicrobial with a bone growth promoter, can be applied by the orthopedic surgeon through the cannulated sections of the bone screw (FIG. 6A) or bolt (FIG. 6B) which perfuses through the plurality of apertures at the closed bore end to inhibit biofilm formation and/or kills the bacteria responsible for infection at or near the bore end apertures as shown in FIGS. 6A and 6B.

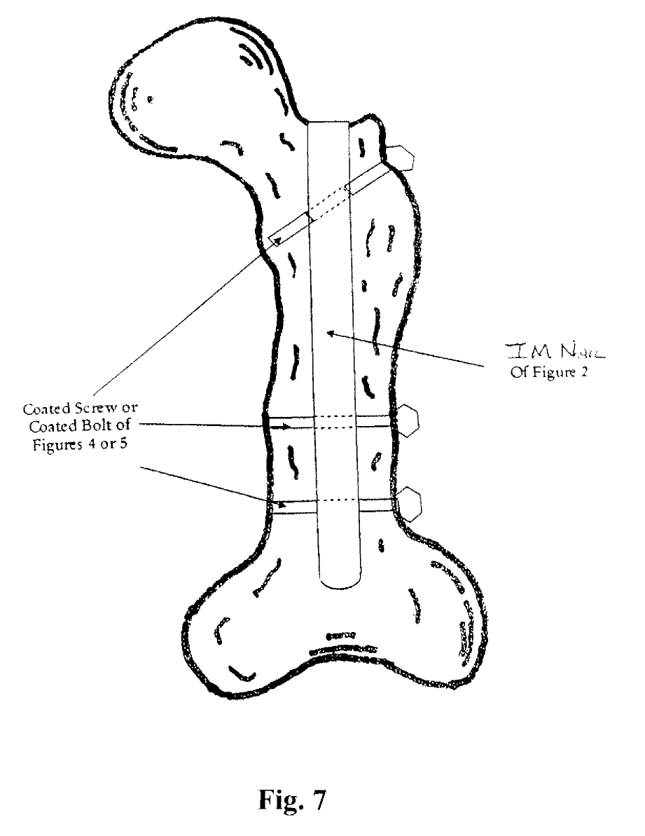

The installation of the coated fracture fixation system in FIG. 7 (shown in a human femur bone) may be accomplished in the following manner: i) upon completion of the usual preliminary work (repositioning, boring, etc.), a peptide-based antimicrobially coated screw of FIG. 5A or bolt of FIG. 5B is inserted in the human leg or arm bone at a predetermined location by the orthopedic surgeon; ii) the peptide-based antimicrobially coated IM nail of FIG. 3 is introduced into the proximal insert opening of the medulla bore; iii) the slotted portion of the coated IM nail engages the previously inserted coated screw of FIG. 4A or bolt of FIG. 4B and seats to the position determined by the orthopedic surgeon; iv) the remaining coated screws of FIG. 4A or bolt of FIG. 4B are inserted in their predetermined locations by the orthopedic surgeon; and v) the peptide-based antimicrobial or novel composite material can be introduced by the orthopedic surgeon through the cannulated sections of the bone screw or bolt and allowed to perfuse through the plurality of apertures at the closed bore ends as shown in FIGS. 6A and 6B, respectively.

In other embodiments internal and external components of tubes that enter a patient's body are coated with peptide-based antimicrobial. Preferably, the antimicrobial peptide is an alpha helical peptide. More preferably, the antimicrobial peptide is selected LLPs, LBUs and eCAPs. More preferably, the antimicrobial peptide is selected from SEQ ID NO. 1 through SEQ ID NO. 185. Preferred antimicrobial peptides to be used as antimicrobial coatings on tubes, include but are not limited to, SEQ ID NO. 4, SEQ ID NO. 5, SEQ ID NO. 8, SEQ ID NO. 9, SEQ ID NO. 173 through SEQ ID NO. 185. The antimicrobial peptide may be immobilized or temporarily attached to the surface of the implant or device.

Embodiments include, but are not limited to antimicrobial peptide coatings on endotracheal tubes, nasogastric tubes, catheters, and shunts. Any tubular structure that penetrates body tissue or fluid of a patient and are coated with the antimicrobial peptides disclosed herein are encompassed in embodiments of this invention. Embodiments include tubes that are uniformly coated with antimicrobial peptide based coatings disclosed herein on the external surface, the internal surface, or both. In another embodiment, the, tubes may only be partially coated on the inside or the outside of the tube, or both. Any combinations of partial or uniform coatings on the inside or the outside of tubes, or both are encompassed in the embodiments of the present invention. The immobilized peptide-based on the aforementioned medical devices kills bacteria introduced during the intubation system; prevents bacterial migration into the body through these components; and prevents or disrupts bacterial biofilm formation and/or kills the bacteria responsible

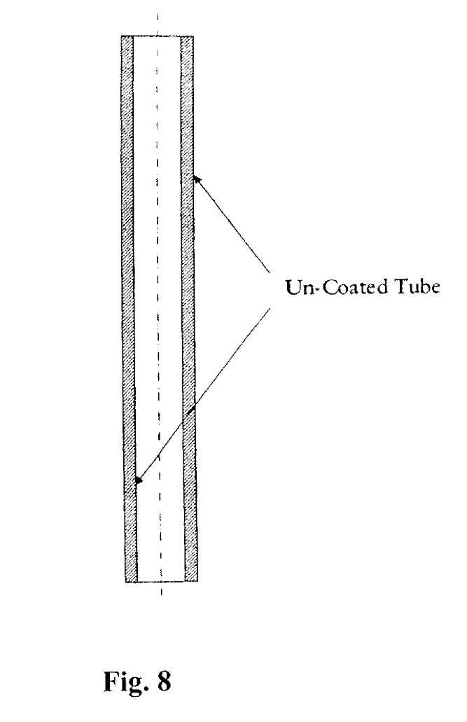

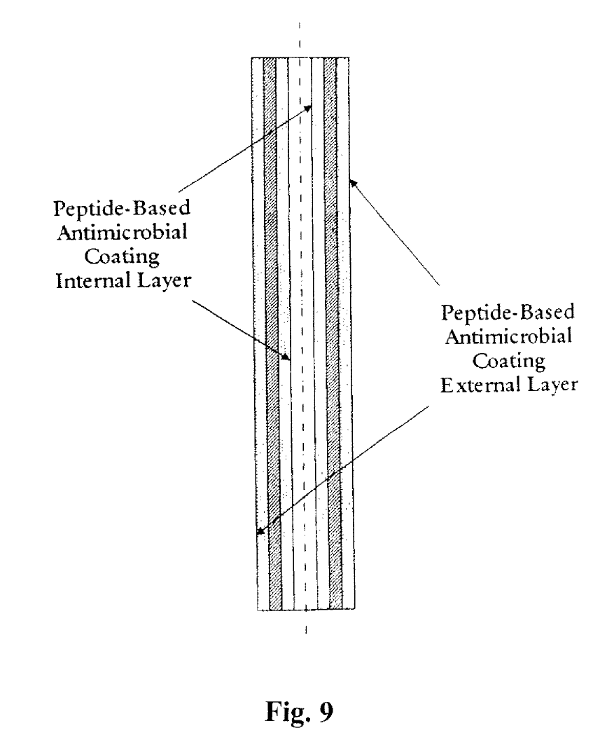

In embodiments, a tube may be uniformly coated with an antimicrobial peptide that is immobilized in a fashion disclosed hereinabove. For example, a tube that is coated with an immobilized antimicrobial peptide is depicted in FIG. 9. The tube can be made of flexible or rigid, clear or translucent, and reinforced or unreinforced materials. Its preferred embodiment is flexible with sufficient rigidity to allow for advancement without excessive force and of varying diameter to accommodate variations in patient's size and anatomy, as shown in FIG. 8. It can be made from non-toxic, medical grade polymers, such as, but not limited to polyvinyl chloride (PVC), rubber, latex, silicone or other similar suitable medical grade materials for human or mammalian use.

The tube is internally and externally substantially uniformly coated with an antimicrobial peptide coating that is immobilized on the tube surfaces as described hereinabove, and depicted in FIG. 9. In a preferred embodiment, the peptide is covalently bonded to the tube surface. The tube can be used as a component to an endotracheal apparatus for intubating a patient's airway, catheter, or shunt. The tube's coating can kill bacteria introduced by the intubation procedure, prevent or disrupt biofilm formation along the tube, and generally prevent the migration of bacteria along the tube, leading to improved patient outcome.

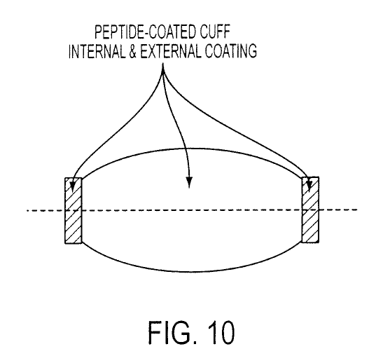

The cuff is internally and externally substantially uniformly coated with an antimicrobial peptide that is immobilized on the tube surfaces as described hereinabove, and depicted in FIG. 10. In a preferred embodiment, the peptide is covalently bonded to the cuff surface. It can be used as a component to an endotracheal apparatus for intubating a patient's airway. The cuff's coating can kill bacteria introduced by the intubation procedure, prevent or disrupt bacterial biofilm formation within the cuff, and generally prevent migration of bacteria into the lung, leading to an improved patient outcome.

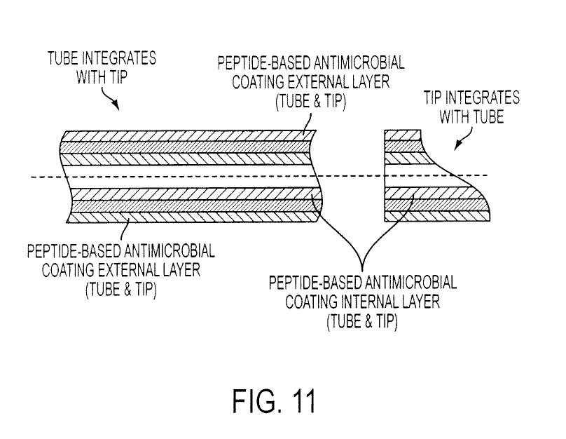

The tip is internally and externally substantially uniformly coated with an antimicrobial peptide that is immobilized on the tube surfaces as described hereinabove, and depicted in FIG. 11. In a preferred embodiment, the peptide is covalently bonded to the tip surface. It can be used as a component to an endotracheal apparatus for intubating a patient's airway, catheter, or shunt. The tip's coating can kill bacteria introduced by the tip, and generally prevent the migration of bacteria within the tip, leading to improved patient outcome.

The coated tube, cuff, and tip shown in the figures and described herein can be used for endotracheal apparatus used for intubating a patient's airway. Another application of the instant invention is for antimicrobial coatings on nasogastric tubes associated with enteral feeding of a patient. Another application can be antimicrobial coated tubes and associated components for catheters (venous, urinary, and others) or ventricular shunts.

Embodiments of the present invention are directed to bonding and immobilizing antibacterial peptides to biocompatible material surfaces of permanent or semi-permanent implantable biomedical devices for mammals. Permanent or semi-permanent implantable biomedical devices may include but are not limited to for example bone fracture fixation systems, shunts, stents, monitoring sensors, cardiac assist devices, penile implants, and the like. In other embodiments, the antibacterial peptides may be bonded and immobilized on surfaces of biomedical devices that may invade the normal body barriers for only a therapeutic time. Other such devices include but are not limited to tubes such as endotracheal, nasogastric, and feeding tubes, for example, dialysis lines, and indwelling catheters, such as Foley and central venous catheters, for example.

The antimicrobial peptides of the coatings of the present invention can also be administered in combination with other active ingredients, such as, for example, bone growth promoters, adjuvants, protease inhibitors, or other compatible drugs or compounds where such combination is seen to be desirable or advantageous in achieving the desired effects of the methods described herein. These additional active ingredients or agents may be combine with the antimicrobial peptide or may be a second layer coating the device.

In embodiments, the immobilized antimicrobial peptides on biomedical device surfaces may prevent the formation of a biofilm on the biomedical device surface. A biofilm is an excellent growth medium for bacteria and precipitates infection. By prevention of bacterial colonization of the biomedical device surface, any bacteria remaining in the subjects system are accessible for clearance by the patient's immune system.

A biocompatible material (or biomaterial) is a synthetic or natural material used to replace part of a living system or to function in intimate contact with living tissue. Biocompatible materials are intended to interface with biological systems to evaluate, treat, augment or replace any tissue, organ or function of the body. Biocompatible materials may include for example synthetic and natural polymers, such as but not limited to acrylic, polyethylene terephthalate (PET), polymethyl methacrylate (PMMA), polypropylene resins cellulose and collagen. Metals with high strength, low modulus and body fluid resistance, such as, but not limited to titanium and titanium alloys are considered biocompatible materials. For biomedical devices that require flexibility, metals such as nitinol, stainless steel, and cobalt chromium are exemplary biomaterials encompassed in embodiments herein. Any biomaterials known now or hereafter to one of ordinary skill in the art are encompassed in embodiments herein.

The antimicrobial peptides may be bonded directly to at least a portion of a biocompatible surface of a biomedical device, or alternatively may be bonded to linking groups that are bonded to at least a portion of the biocompatible surface. For immobilization of the antimicrobial peptide on the biomedical device surface, covalent bond formation between the surface and the peptide; or between the surface, the linking group, and the peptide is preferred. Other types of bonding, including, but not limited to acid-base interactions, ionic bonding, hydrogen bonding, dispersion forces, and van der Waals interactions are included in the scope of embodiments herein.

The term “immobilized” as used herein refers to antibacterial peptides that remain bonded to the surface of the device, and maintain efficacy for at least a therapeutic time period. The peptide remains bonded and active as a bactericide at least until the chance for bacterial infection is not elevated as compared to the condition of the patient's normal physiology. For embodiments that include permanent or semi-permanent implantable biomedical devices, the peptide may remain bonded to the surface of the device after the threat of infection has passed in order to promote the adhesion and maturation of cells on the implanted device.

In embodiments where the antibacterial peptide is bonded directly to the surface of the biomedical device the biomedical device may be cleaned or activated to increase the polarity of the surface, or to provide functional groups on the surface, to promote chemical reaction of the peptide and the surface. Cleaning, passivating, or activating a surface may include chemical processes such as, but not limited to, repetitive acid or alkaline washing followed by rinsing with distilled or deionized water. Cleaning, passivating, or activating a surface may also include physical processes such as for example corona discharge. Any cleaning and/or activating process known now or hereafter to one of ordinary skill in the art is within the scope of embodiments herein.

In embodiments, a linking group, also known as a linker, a coupling agent, a primer, and a tie layer, is used to bond an antimicrobial peptide to a device surface is a form of chemical surface functionalization. One end of the linking group readily forms bonds that are stable under physiological conditions with the device surface. The linking group may then be capable of forming hydrolytically and physiologically stable bonds with a terminal end of an antimicrobial peptide. In other embodiments, after the surface is functionalized with a linking group, the linking group may be further treated to create reactive sites on the linking group. Such treatments may include corona discharge, plasma processes, flame treatments, or other processes known now or hereafter to one of ordinary skill in the art to create polar and/or reactive groups, such as for example, hydroxylcarboxyl and carbonyl, on a non-polar linking group. In this fashion, the terminal ends of the antimicrobial peptide can chemically bond with the linking group.

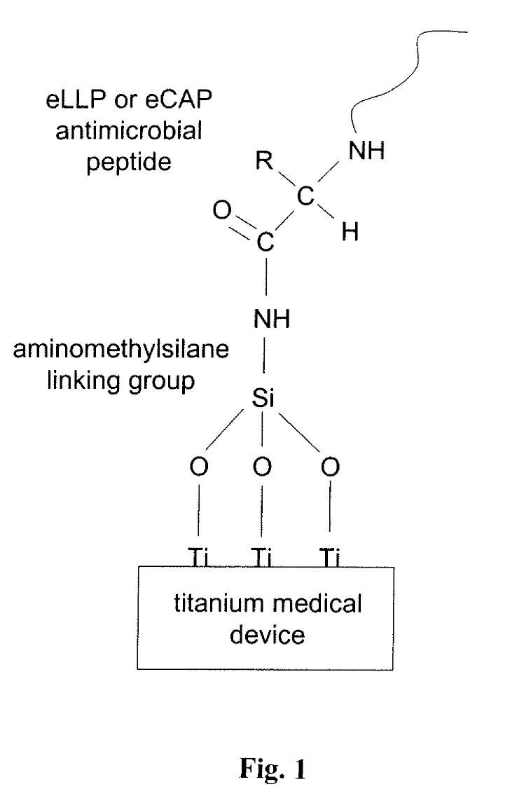

In one embodiment, silanization may be used to functionalize the surface of a biomedical device. A schematic diagram of a non-limiting embodiment of the present invention where a portion of a peptide is bonded to the surface of a medical device made of titanium is shown in FIG. 1. The device surface is silanized using an organosilane which has an organic group that may be used to couple other molecules to the device surface. The organic group may include but is not limited to one or more amine groups, epoxides, carboxylic acids, thiols, or vinyl groups. Titanium metal has an oxidized surface. The outermost layer may consist of hydroxyl groups. The surface hydroxyl groups can react with the organosilane to form Ti—O—Si hydrolytically stable bonds. In the non-limiting example of FIG. 1, an organosilane such as aminomethyltriethoxysilane is reacted with the titanium surface. The C-terminus group of the peptide can then react with the amino group on the silanized surface. In addition, a network of silane can be formed by the reaction of silanol groups of different molecules resulting in greater stability of the organosilane layer. The hydrolyzable bond on an organosilane can be alkoxy bonds, such as methoxy and ethoxy, or it could be a chlorine side group. The fourth group is selected for reaction with the antimicrobial peptide. In the example of FIG. 1, an amino group is shown to react with the carboxylic acid group of an antimicrobial peptide. Similarly, the fourth group of the organosilane could be a carboxylic acid group to react with the N-terminus group of the peptide. A photoactivatable group, such as for example, benzophenone can be used. In embodiments where hydrophobic interaction of the hydrophobic portions of an antimicrobial peptide with a hydrophobic surface of a medical device is desirable, an organosilane such as octadecyltrimethoxysilane may be used.

In other embodiments, a material such as a block copolymer may be used to functionalize a surface of a biomedical device with antimicrobial proteins. In these instances, a hydrophobic block of a copolymer may interact with a hydrophobic device surface. A hydrophilic block of the copolymer may extend away from the surface, and can have functional groups that can react with the surface.

In other embodiments, a parylene coating may be initially applied to the medical device surface. Parylene is the tradename for a variety of polyxylylene polymers marketed by Para Tech Coating, Inc., Aliso Viejo, Calif. Parylene C is a polymer manufactured from di-p-xylene, a dimer of p-xylene. Di-p-xylene, more properly known as [2.2]paracyclophane, is made from p-xylene in several steps involving bromination, amination and elimination. Heating [2.2]paracyclophane in a partial vacuum gives rise to a diradical species, which polymerizes when deposited on a surface. Until the “monomer” comes into contact with a surface it is in a gaseous phase and can access the entire exposed surface. A biomedical device with a parylene coating can then be further treated to produce polar reactive groups at the surface, so that antimicrobial peptides can chemically react with the modified parylene coated surface. Methods to produce polar reactive groups on plastic and metallic materials are known in the art, and may include for example ozone treatments, corona discharge, flame treatments, plasma processing, acid etching, ultraviolet light irradiation, gamma ray irradiation, and electron beam irradiation. It is recognized that these surface modification techniques can be used on most polymeric surfaces to produce reactive groups to which the antimicrobial peptides can form covalent bonds and become immobilized on the medical device surface.

Embodiments of using the antimicrobial peptides disclosed and claimed herein include making composite coatings of polymers with the antimicrobial peptides. For example a rubber surface can be modified by photochemical immobilization of an antimicrobial peptide. A photochemically reactive compound can be made to react with a surface using the appropriate wavelength of light. Antimicrobial peptides can then be reacted with the photochemical reactive compound, and thus immobilizing the peptide on the device surface. United States Patent Publication 2003/0228410 discloses a photochemical process to immobilize biomolecules on a surface using a photochemical reaction of 1-fluoro-2-nitro-4-azidobenzene with the polymer surface. Generally, any polymer having a carbon-hydrogen bond can be photochemically activated. The antimicrobial peptides disclosed and claimed herein can the react with the photochemically activated surface. Other methods of photochemically immobilizing biomolecules known now or hereafter to one of ordinary skill in the art are within the scope of embodiments disclosed and claimed herein.

In a further embodiment, another method to immobilize antimicrobial peptides of the instant invention is through ionic bonding. For example, a charged surface active agent may react with the surface and act as an anchor for chemically reacting with the antimicrobial peptides. For example tri decyl methyl ammonium chloride (TDMAC) is a surfactant used for binding antibiotics on medical device surfaces. TDMAC has a quaternary ammonium compound with three long hydrophobic chains and a positively charged nitrogen. A negatively charged end of an antimicrobial peptide zwitterion of those disclosed and claimed in this invention could ionically bond with the positively charged nitrogen, and thus be anchored or immobilized on the device surface.

Entrapment of antimicrobial peptides in a polymer matrix that is applied on a medical device surface is another means of preparing an antimicrobial coating in embodiments of the present invention. Application of non-reactive polymer matrices is used for indwelling medical devices. Polymer matrices with antimicrobial peptides could be used to construct a device or applied as a coating on a device. In embodiments, the antimicrobial peptides may not be chemically reacted with the polymer matrix. The peptides may then slowly diffuse out of the matrix and work as a bactericide in the vicinity of the surface. A sustained release of the antimicrobial peptides is possible, resulting in longer term protection from infection. An exemplary, but not limiting, polymer matrix is one of polyurethane.

In useful embodiments of the present invention it may be desirable for antimicrobial peptides to both be covalently bonded to the surface of a medical device and to exhibit a sustained release or elution from the surface of a medical device. In such scenarios, the total of the eluting phase and the covalently bonded phase provides protection and/or treatment in the first instances after insertion of the medical device. As the eluting phase is depleted the covalently bonded phase may provide long term protection or protection from future infections.

For example, an antimicrobial peptide may be provided to the treated surface of a medical in excess of what can covalently bond with the surface. The excess may be entrapped in a polymeric matrix of the surface treatment, or alternatively, or in addition, otherwise bonded through hydrogen bonding or dispersion forces between the peptides, for example.

Other means of treating surfaces or depositing coatings on surfaces such as plasma vapor deposition (PVD), chemical vapor deposition (CVD), graft polymerization are also techniques that can be used to immobilize antimicrobial peptides on a medical device surface. Any surface modification technique that is known now or hereafter to one or ordinary skill in the art, which can be used to enable covalent bonding or other method of immobilization of antimicrobial peptides on a medical device surface, or to facilitate elution of antimicrobial peptides from a medical device, are within the scope of this invention.

The antimicrobial peptides of the present invention are peptides which exhibit antimicrobial activity against diverse microorganisms. Preferably the antimicrobial peptides are alpha-helical, more preferably the antimicrobial peptides are selected from groups designated herein as LLPs, LBUs, and eCAPs. The LLPs, LBUs, and eCAPs classes of peptides are further disclosed and described below. In one aspect of the invention, the peptides correspond to amino acid sequences in the transmembrane (TM) proteins of lentiviruses, in particular, HIV and SIV. These peptides comprise arginine-rich sequences, which, when modeled for secondary structure, display high amphipathicity and hydrophobic moment. The antimicrobial peptides are highly inhibitory to microorganisms but significantly less toxic to red blood cells and other normal mammalian cells. As a result, these peptides can be characterized as selective antimicrobial agents.

In a useful embodiment of the present invention, the antimicrobial peptides may retain antibacterial activity after a device coated with the antimicrobial peptide is sterilized. It is reported below that the WLBU-2 peptide coated on a substrate retained antimicrobial activity after ethylene oxide sterilization. Preliminary experiments on neat solid antimicrobial peptides disclosed herein or liquid solutions of peptides disclosed herein have demonstrated that several of the peptides remained biologically active after low and high gamma-irradiation, and high temperature sterilization processes.

This invention and embodiments illustrating the method and materials used may be further understood by reference to the following non-limiting examples.

### EXAMPLE 1

Medical standard plastic endodontic obturators were coated with an antimicrobial peptide. The obturators had a layer of gutta-percha over ⅔ the length of the shaft. The obturators were coated with a tie layer. A parylene coating containing a reactive molecule specific for the N-terminus region of the peptide (“Photopolymer”) was then applied over the tie layer. The peptide WLBU-2 (SEQ ID NO. 179) was applied to the parylene coating and exposed to UV light to facilitate covalent bonding of the peptide with the parylene coating. Non-coated obturators were used as controls. All samples and controls were subjected to ethylene oxide sterilization. The coated samples were placed in 75% relative humidity overnight. The immobilized antimicrobial peptide was tested in an in-vitro assay system showing attachment of the antimicrobial peptide to the plastic implant and efficacy against Enterococcis faecalis (data not shown).

### EXAMPLE 2

Medical standard alloy 316L stainless steel pins were coated with an antimicrobial peptide layer to determine the efficacy of an immobilized antimicrobial peptide. The pins were coated with a tie layer. A parylene coating containing a reactive molecule specific for the N-terminus region of the peptide (“Photopolymer”) was then applied over the tie layer. The peptide WLBU-2 (SEQ ID NO. 179) was applied to the parylene coating and exposed to UV light to facilitate covalent bonding of the peptide with the parylene coating. Parylene coated stainless steel pins and non-coated stainless steel pins were used as controls. All samples and controls were subjected to ethylene oxide sterilization. The coated samples were placed in 75% relative humidity overnight.

### EXAMPLE 3

The pins of Example 2 were exposed to Staphylococcus aureus (University of Calgary clinical isolate, strain 2654) using an in-vitro assay system outlined below.

An in-vitro assay system for evaluating adhesion, post adhesion killing and inhibition of biofilm formation on surfaces is outlined below. Using a cryogenic stock (−70° C.), a first sub-culture was streaked out of the bacterial organisms listed above on TSA. The plate was incubated at 37±1° C. for 24 hours in 5.0% CO₂ and the plate was stored wrapped in parafilm at 4° C. From the first sub-culture, a second sub-culture was streaked out on TSA and incubated at 37±1° C. for 24 hours in 5.0% CO₂. The second sub-culture was used within 24 hours starting from the time it was first removed from incubation. Using the second sub-culture an inoculum was created in 3 mL sterile water that matched a 0.5 McFarland standard (1.5×10⁸ cells per mL) in a glass test tube using a sterile cotton swab. The inoculum was adjusted to an approximate cell density of 10⁶ CFU/ml in 10% Mueller Hinton broth in PBS and 20 ml of the inoculum was placed in the wells of the EO-BEST Device. A growth control with no stainless steel was made as well. The lid with the attached samples was inserted into the inoculum and the entire device was incubated at 37±1° C. for 24 hours in 5.0% CO₂. After the 24 hours, the samples on the lid are rinsed three times in sterile saline by dipping the lid with samples attached into three consecutive bottom plates containing 23 ml of sterile saline in each well. The lid with samples attached was placed into wells containing 23 ml of sterile saline for 2 hours to allow recently adhered bacteria to be acted upon by any antimicrobial in the coating. The EO-BEST lid was then inserted into wells containing 23 ml of sodium thioglycollate (0.1% w/v) (Fisher Scientific) and 0.5% Tween 80 (Fisher Scientific) in phosphate buffered saline (PBS) in each well. The entire device assembled above was sonicated for 30 minutes. Following sonication, 100 μl from each well of the EO-BEST plate was placed into the first 12 empty wells of the first row of a 96 well-micro titer plate. 180 μl of 0.9% sterile saline was placed in the remaining rows. A serial dilution (100-105) was prepared by moving 20 μl down each of the 8 rows. 20 μl was removed from each well and spot plated on a prepared Trypticase Soy Agar plate. Plates were incubated at 37±1° C. in 5.0% CO₂ and counted after approximately 24 hours of incubation.

CFU total (per vial) of Staphylococcus aureus (University of Calgary clinical isolate, strain 2654) on WLBU-2 (SEQ ID NO. 179) coated photopolymer pins, on photopolymer-only coated pins, and non-coated (control) pins is found the following Table 4.

TABLE 4

| | Sample | Internal Replicate (LOG 10 CFU) | | |
|---|---|---|---|---|
| Sample | Description | 1 | 2 | 3 |
| C | Control | 3.92 | 3.2 | 3.39 |
| B | Photopolymer | 4.63 | 4.43 | 4.20 |
| A | Peptide | 0.00 | 0.00 | 0.00 |

There was significantly reduced growth (no bio-film growth at all) of Staphylococcus aureus on the peptide coated-photopolymer coated pins, compared to the photopolymer-only coated pins and the un-coated control pins, under the test conditions of this experiment.

The peptide/photopolymer coated pins were effective in preventing biofilm adherence, and preventing biofilm formation compared to the uncoated and photopolymer-only coated pins, under the test conditions of this experiment.

### EXAMPLE 4

The procedure provided describes an assay for testing 2 organisms (S. epidermidis and S. aureus) grown as a biofilm against 2 antimicrobial agents (gentamicin and an eCAP peptide) using a 96 well microtitre plate. The procedure will allow one to compare the MIC and MBEC values for each organism against each antimicrobial agent and against each other. Each test included the following groups listed in Table 5 below.

TABLE 5

| Code | Sample | Description |
|---|---|---|
| A | Gentamicin (Control) | Serial diluted from 3.0 μmol/mL |
| | | to 0.0029 μmol/mL |
| B | eCAP (WLBU2) | Serial diluted from 1.0 μmol/mL |
| | | to 0.00098 μmol/mL |
| C | Gamma Radiated eCAP | Serial diluted from 1.0 μmol/mL |
| | Low Dose (WLBU2) | to 0.00098 μmol/mL |
| D | Gamma Radiated eCAP | Serial diluted from 1.0 μmol/mL |
| | High Dose (WLBU2) | to 0.00098 μmol/mL |
| E | Ethylene Oxide Exposed | Serial diluted from 1.0 μmol/mL |
| | eCAP (WLBU2) | to 0.00098 μmol/mL |

Materials.

Universal Neutralizer (for biocide testing): 1.0 g L-Histidine; 1.0 g L-Cysteine; 2.0 g Reduced glutathione; Made up to 20 ml in double distilled water; Passed through a syringe with a 0.20 μm filter to sterilize. This solution was stored at −20° C.

Made up 1 liter of the appropriate growth medium (TSB). Supplemented this medium with 20.0 g per liter of saponin and 10.0 g per liter of Tween-80. Adjusted with dilute NaOH to the correct pH (7.0±0.2 at 20° C.).

Added 500 μl of the universal neutralizer to each 20 ml of the surfactant supplemented growth medium used for recovery plates.

Microorganisms used were Staphylococcus epidermididis (RP62A; “Christensen Strain”) and Staphylococcus aureus (University of Calgary clinical isolate, strain 2645).

Preparation of Antibiotic Challenge Plate: As set forth in Test Panels 1, 2 and 3, which are described in Tables 6, 7, and 8 respectively.

Test Panel 1:

TABLE 6

| | 1 | 2 | 3 | 4 | 5 | 6 | 7 | 8 | 9 | 10 | 11 | 12 |
|---|---|---|---|---|---|---|---|---|---|---|---|---|
| A | | | | | | | | | | | | |
| B | 3.0 | 1.5 | 0.75 | 0.38 | 0.19 | 0.094 | 0.047 | 0.023 | 0.011 | 0.0059 | 0.0029 | GC |
| C | 3.0 | 1.5 | 0.75 | 0.38 | 0.19 | 0.094 | 0.047 | 0.023 | 0.011 | 0.0059 | 0.0029 | GC |
| D | 3.0 | 1.5 | 0.75 | 0.38 | 0.19 | 0.094 | 0.047 | 0.023 | 0.011 | 0.0059 | 0.0029 | GC |
| E | 1.0 | 0.5 | 0.25 | 0.13 | 0.063 | 0.031 | 0.016 | 0.0078 | 0.0039 | 0.0020 | 0.00098 | GC |
| F | 1.0 | 0.5 | 0.25 | 0.13 | 0.063 | 0.031 | 0.016 | 0.0078 | 0.0039 | 0.0020 | 0.00098 | SC |
| G | 1.0 | 0.5 | 0.25 | 0.13 | 0.063 | 0.031 | 0.016 | 0.0078 | 0.0039 | 0.0020 | 0.00098 | SC |
| H | | | | | | | | | | | | SC |

Rows A-D, Columns 1-11 = gentamicin
Rows E-H, Columns 1-11 = eCAP

### Test Panel 2:

TABLE 7

| | 1 | 2 | 3 | 4 | 5 | 6 | 7 | 8 | 9 | 10 | 11 | 12 |
|---|---|---|---|---|---|---|---|---|---|---|---|---|
| A | | | | | | | | | | | | |
| B | 1.0 | 0.5 | 0.25 | 0.13 | 0.063 | 0.031 | 0.016 | 0.0078 | 0.0039 | 0.0020 | 0.00098 | GC |
| C | 1.0 | 0.5 | 0.25 | 0.13 | 0.063 | 0.031 | 0.016 | 0.0078 | 0.0039 | 0.0020 | 0.00098 | GC |
| D | 1.0 | 0.5 | 0.25 | 0.13 | 0.063 | 0.031 | 0.016 | 0.0078 | 0.0039 | 0.0020 | 0.00098 | GC |
| E | 1.0 | 0.5 | 0.25 | 0.13 | 0.063 | 0.031 | 0.016 | 0.0078 | 0.0039 | 0.0020 | 0.00098 | GC |
| F | 1.0 | 0.5 | 0.25 | 0.13 | 0.063 | 0.031 | 0.016 | 0.0078 | 0.0039 | 0.0020 | 0.00098 | SC |
| G | 1.0 | 0.5 | 0.25 | 0.13 | 0.063 | 0.031 | 0.016 | 0.0078 | 0.0039 | 0.0020 | 0.00098 | SC |
| H | | | | | | | | | | | | SC |

Rows A-D, Columns 1-11 = eCAP sterilized with Low Dose Gamma Radiation
Rows E-H, Columns 1-11 = eCAP sterilized with High Dose Gamma Radiation

### Test Panel 3:

TABLE 8

| | 1 | 2 | 3 | 4 | 5 | 6 | 7 | 8 | 9 | 10 | 11 | 12 |
|---|---|---|---|---|---|---|---|---|---|---|---|---|
| A | | | | | | | | | | | | |
| B | 1.0 | 0.5 | 0.25 | 0.13 | 0.063 | 0.031 | 0.016 | 0.0078 | 0.0039 | 0.0020 | 0.00098 | GC |
| C | 1.0 | 0.5 | 0.25 | 0.13 | 0.063 | 0.031 | 0.016 | 0.0078 | 0.0039 | 0.0020 | 0.00098 | GC |
| D | 1.0 | 0.5 | 0.25 | 0.13 | 0.063 | 0.031 | 0.016 | 0.0078 | 0.0039 | 0.0020 | 0.00098 | GC |
| E | | | | | | | | | | | | GC |
| F | | | | | | | | | | | | SC |
| G | | | | | | | | | | | | SC |
| H | | | | | | | | | | | | SC |

Rows A-D, Columns 1-11 = cCAP sterilized with Ethylene Oxide

One set of test panels as shown above was prepared for each organism to determine the MIC and MBEC after a 24 hour exposure to each test material. SC wells are sterility controls for each experiment. GC is the growth control for each row. The working solution of gentamicin was 3 μmol/ml and 1 μmol/ml for eCAP. No SEM samples were taken for analysis due to test material shortage.

Using a sterile 96-well microtitre plate under the laminar flow hood, the following was done to set up the above challenge plate for 1 organism per plate:

Sample sterility controls: Pegs F12-H12 were broken off with flamed pliers. Each peg into was placed in 200 uL of the neutralizer (described in section 4.1) in a 96 well plate. The pegs were sonicated for 15 minutes. Samples were serially diluted and spot plated on TSA. This serves as a biofilm growth check.

A working solution of eCAP or Gentamicin (1 μMoles/mL and 3 μMoles/mL respectively) was made in 10% MHB in sterile PBS. 200 μl of 10% MHB was added to ‘column’ 12 to serve as sterility controls (SC) and growth controls (GC) for each organism. 100 μl of 10% MHB was added to ‘columns’ 2 to 11. 200 μl of the appropriate working solution was added to ‘column’ 1 of the microtitre plate. 100 μl of the appropriate working solution was added to ‘column’ 2 and ‘column’ 3 of the microtitre plate. Using the multichannel micropipette, the contents of ‘column’ 3 was mixed by pipetting up and down. After mixing, 100 μl was transferred from the wells in ‘column’ 3 to the corresponding wells in ‘column’ 4. This was repeated until reaching ‘column’ 1. Using a pipet, 100 μl from each well in ‘column’ 11 was ejected. 100 μl of growth media was then added to all wells except A1 to H1, A2 to H2, and A12 to H12.

Antimicrobial Challenge of Biofilm: Rinse plate(s) of saline (200 μL per well) were prepared. Planktonic cells from biofilm that had formed on the lid of the MBEC device were rinsed by dipping the lid into the saline for 1-2 minutes. The lid was transferred to the challenge plate and incubated at 37° C. for 24 hours on a gyrating platform set at approximately 150 rpm.

Determination of Planktonic MIC: The challenge plate was incubated at 37° C. for 24 hours and read using a plate reader to determine MIC values. The MIC (minimum inhibitory concentration) was determined for compound eCAP/gentamicin for each organism shed from the biofilm during the challenge incubation. The MIC is defined as the minimum concentration that inhibits growth of the organism.

Recovery of Surviving Biofilm: Rinse plate(s) of 0.9% sterile saline (200 μL per well) were prepared in a sterile 96 well micro titre plate. Pegs were rinsed in 0.9% sterile saline for 1 to 2 minutes. Recovery plate(s) of neutralizer described in section 4.1 (200 μL per well) were prepared in another 96 well micro titre plate. Pegs were transferred to recovery media then sonicated on high for 15 minutes to dislodge surviving biofilm. The plate(s) were placed in a dry stainless steel insert tray which sits in the water of the sonicator. The vibrations created in the water by the sonicator transfer through the insert tray to actively sonicate the contents of the 96 well recovery plate(s). The recovery plates were incubated at 37° C. for 24 hours and read using the plate ready to determine MBEC values.

Results: The proof of concept pre-study demonstrated that the compound eCAP forms a very turbid suspension when diluted in TSB and indicated that the TSB may affect the activity of eCAP. Therefore, 10% MHB was used as a diluent instead of TSB. When the test organisms were grown in 10% MHB in PBS, they demonstrate a low turbidity for the MIC test. Therefore, a substantial amount of growth is considered to be ≧0.075 for S. aureus and ≧0.045 for S. epidermidis. For the MBEC test, outliers were present for S. epidermidis and S. aureus which is common in broth dilution assays. There is a greater amount of outliers for S. aureus and for eCAP sterilized by ethylene oxide and irradiation. The MIC and MBEC values were determined as the dilution immediately before the concentration where growth was first observed, as depicted in Table 9 below.

Staphylococcus aureus: The MIC value for eCAP and eCAP sterilized by all 3 tested methods is between 0.0020 μmol/ml and 0.00098 μmol/ml. The MIC value for Gentamicin was less than 0.0029. The MBEC value for Gentamicin is 1.5 μmol/ml. The MBEC value for eCAP is between 0.5 μmol/ml and 0.25 μmol/ml. The MBEC value for eCAP sterilized by low dose irradiation is approximately 0.063 μmol/ml. The MBEC value for eCAP sterilized by high dose irradiation is approximately 0.016 μmol/ml. The MBEC value for eCAP sterilized by ethylene oxide is approximately 0.0039 μmol/ml.

Staphylococcus epidermidis: The MIC value for eCAP and eCAP sterilized by ethylene oxide is 0.0039 μmol/ml and the value for eCAP sterilized by high and low dose irradiation is between 0.0039 μmol/ml and 0.0020 μmol/ml. The MIC value for Gentamicin is 1.5 μmol/ml. The MBEC value for eCAP is between 0.0078 μmol/ml and 0.0039 μmol/ml for S. epidermidis. The MBEC value for eCAP sterilized by ethylene oxide is 0.0078 ∞mol/ml. The MBEC value for the high and low dose irradiated eCAP is between 0.016 μmol/ml and 0.0078 μmol/ml. The MBEC value for Gentamicin is higher than 3.0 μmol/ml.

It appears that the sterilization methods (high and low dose gamma radiated and ethylene oxide) for eCAP are not detrimental to its antimicrobial activity.

TABLE 9

| | S. aureus | | S. epidermidis | |
|---|---|---|---|---|
| | MIC | MBEC | MIC | MBEC |
| | (μmol/ml) | (μmol/ml) | (μmol/ml) | (μmol/ml) |
| Gentamicin | <0.0029 | 1.5 | 1.5 | >3.0 |
| eCAP | 0.0020-0.00098 | 0.5-0.25 | 0.0039 | 0.0078-0.0039 |
| High dose Irradiated eCAP | 0.0020-0.00098 | ~0.016 | 0.0039-0.0020 | 0.016-0.0078 |
| Low dose irradiated eCAP | 0.0020-0.00098 | ~0.063 | 0.0039-0.0020 | 0.016-0.0078 |
| Ethylene oxide eCAP | 0.0020-0.00098 | ~0.0039 | 0.0039 | 0.0078 |

Although the present invention has been described in considerable detail with reference to certain preferred embodiments thereof, other versions are possible. Therefore the spirit and scope of the appended claims should not be limited to the description and the preferred versions contained within this specification.

What is claimed:

1. An implantable article comprising:
an implant for a mammal having a biologically compatible surface, wherein at least a portion of the biologically compatible surface further includes a therapeutically effective amount of an antimicrobial peptide selected from the group consisting of a linear lytic peptide, a lytic base unit peptide and an engineered cationic antimicrobial peptide.

2. The implantable article of claim 1, wherein the linear lytic peptide is selected from the group consisting of SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15, SEQ. ID NO. 16, SEQ. ID NO. 17, SEQ. ID NO. 18, SEQ. ID NO. 19, SEQ. ID NO. 20, SEQ. ID NO. 21, SEQ. ID NO. 22, SEQ. ID NO. 23, SEQ. ID NO. 24, SEQ. ID NO. 25, SEQ. ID NO. 26, SEQ. ID NO. 27, SEQ. ID NO. 28, SEQ. ID NO. 29, SEQ. ID NO. 30, SEQ. ID NO. 31, SEQ. ID NO. 32, SEQ. ID NO. 33, SEQ. ID NO. 34, SEQ. ID NO. 35, SEQ. ID NO. 36, SEQ. ID NO. 37, SEQ. ID NO. 38, SEQ. ID NO. 39, SEQ. ID NO. 40, SEQ. ID NO. 41, SEQ. ID NO. 42, SEQ. ID NO. 43, SEQ. ID NO. 44, SEQ. ID NO. 45, SEQ. ID NO. 46, SEQ. ID NO. 47, SEQ. ID NO. 48, SEQ. ID NO. 49, SEQ. ID NO. 50, SEQ. ID NO. 51, SEQ. ID NO. 52, SEQ. ID NO. 53, SEQ. ID NO. 54, SEQ. ID NO. 55, SEQ. ID NO. 56, SEQ. ID NO. 57, SEQ. ID NO. 58, SEQ. ID NO. 59, SEQ. ID NO. 60, SEQ. ID NO. 61, SEQ. ID NO. 62, SEQ. ID NO. 63, SEQ. ID NO. 64, SEQ. ID NO. 65, SEQ. ID NO. 66, SEQ. ID NO. 67, SEQ. ID NO. 68, SEQ. ID NO. 69, SEQ. ID NO. 70, SEQ. ID NO. 71, SEQ. ID NO. 72, SEQ. ID NO. 73, SEQ. ID NO. 74, SEQ. ID NO. 75, SEQ. ID NO. 76, SEQ. ID NO. 77, SEQ. ID NO. 78, SEQ. ID NO. 79, SEQ. ID NO. 80, SEQ. ID NO. 81, SEQ. ID NO. 82, SEQ. ID NO. 83, SEQ. ID NO. 84, SEQ. ID NO. 85, SEQ. ID NO. 86, SEQ. ID NO. 87, SEQ. ID NO. 88, SEQ. ID NO. 89, SEQ. ID NO. 90, SEQ. ID NO. 91, SEQ. ID NO. 92, SEQ. ID NO. 93, SEQ. ID NO. 94, SEQ. ID NO. 95, SEQ. ID NO. 96, SEQ. ID NO. 97, SEQ. ID NO. 98, SEQ. ID NO. 99, SEQ. ID NO. 100, SEQ. ID NO. 101, SEQ. ID NO. 102, SEQ. ID NO. 103, SEQ. ID NO. 104, SEQ. ID NO. 105, SEQ. ID NO. 106, SEQ. ID NO. 107, SEQ. ID NO. 108, SEQ. ID NO. 109, SEQ. ID NO. 110, SEQ. ID NO. 111, SEQ. ID NO. 112, SEQ. ID NO. 113, SEQ. ID NO. 114, SEQ. ID NO. 115, SEQ. ID NO. 116, SEQ. ID NO. 117, SEQ. ID NO. 118, SEQ. ID NO. 119, SEQ. ID NO. 120, SEQ. ID NO. 121, SEQ. ID NO. 122, SEQ. ID NO. 123, SEQ. ID NO. 124, SEQ. ID NO. 125, SEQ. ID NO. 126, SEQ. ID NO. 127, SEQ. ID NO. 128, SEQ. ID NO. 129, SEQ. ID NO. 130, SEQ. ID NO. 131, SEQ. ID NO. 132, SEQ. ID NO. 133, SEQ. ID NO. 134, SEQ. ID NO. 135, SEQ. ID NO. 136, SEQ. ID NO. 137, SEQ. ID NO. 138, SEQ. ID NO. 139, SEQ. ID NO. 140, SEQ. ID NO. 141, SEQ. ID NO. 142, SEQ. ID NO. 143, SEQ. ID NO. 144, SEQ. ID NO. 145, SEQ. ID NO. 146, SEQ. ID NO. 147, SEQ. ID NO. 148, SEQ. ID NO. 149, SEQ. ID NO. 150, SEQ. ID NO. 151, SEQ. ID NO. 152, SEQ. ID NO. 153, SEQ. ID NO. 154, SEQ. ID NO. 155, SEQ. ID NO. 156, SEQ. ID NO. 157, SEQ. ID NO. 158, SEQ. ID NO. 159, SEQ. ID NO. 160, SEQ. ID NO. 161, SEQ. ID NO. 162, SEQ. ID NO. 163, SEQ. ID NO. 164, SEQ. ID NO. 165, SEQ. ID NO. 166, SEQ. ID NO. 167, SEQ. ID NO. 168 and SEQ. ID NO. 169.

3. The implantable article of claim 1, wherein the linear lytic peptide is selected from the group consisting of SEQ ID NO. 4, SEQ ID NO. 5, SEQ ID NO. 8, SEQ ID NO. 9, SEQ ID NO. 10, SEQ ID NO. 38, SEQ ID NO. 73, SEQ ID NO. 92, and SEQ ID NO. 97.

4. The implantable article of claim 1, wherein the lytic base unit peptide is selected from the group consisting of SEQ. ID NO. 170, SEQ. ID NO. 171, SEQ. ID NO. 172, SEQ. ID NO. 173, SEQ. ID NO. 174, SEQ. ID NO. 175, SEQ. ID NO. 176, SEQ. ID NO. 177, SEQ. ID NO. 178, SEQ. ID NO. 179, SEQ. ID NO. 180 and SEQ. ID NO. 181.

5. The implantable article of claim 1, wherein the engineered cationic antimicrobial peptide is selected from the group consisting of SEQ. ID NO. 182, SEQ. ID NO. 183, SEQ. ID NO. 184 and SEQ. ID NO. 185.

6. The article of claim 1, wherein the implant is a fracture fixation system.

7. The article of claim 6, wherein the fracture fixation system is selected from a nail, a bolt and a screw.

8. The article of claim 1, wherein the implant comprises a tubular structure selected from a tube that penetrates a body tissue and a tubular component of an intubation systems.

9. The article of claim 8, wherein the tube that penetrates a body tissue is selected from an intubation tube, a feeding tube, a endotracheal tube, a catheter, and a shunt.

10. The article of claim 1, wherein the antimicrobial peptide is immobilized.

11. The article of claim 1, wherein substantially all of the surface further comprises a therapeutically effective amount of the antimicrobial peptide.

12. The article of claim 1 further comprising a linking layer between the biologically compatible surface and the antimicrobial peptide.

13. A method for immobilizing antimicrobial peptides on a medical device comprising:
providing a medical device;
applying a linking layer to at least a portion of a surface of the medical device; and
reacting an antimicrobial peptide selected from a linear lytic peptide, a lytic base unit peptide and an engineered cationic antimicrobial peptide to the linking layer.

14. The method of claim 13, wherein a method of applying a linking layer is selected from the group consisting of silanization, activated polyxylylene polymer coating, plasma vapor deposition, chemical vapor deposition, surfactant coating; photochemical reactive coating, and block copolymer coating.

15. The method of claim 13, wherein the linear lytic peptide is selected from the group consisting of SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15, SEQ. ID NO. 16, SEQ. ID NO. 17, SEQ. ID NO. 18, SEQ. ID NO. 19, SEQ. ID NO. 20, SEQ. ID NO. 21, SEQ. ID NO. 22, SEQ. ID NO. 23, SEQ. ID NO. 24, SEQ. ID NO. 25, SEQ. ID NO. 26, SEQ. ID NO. 27, SEQ. ID NO. 28, SEQ. ID NO. 29, SEQ. ID NO. 30, SEQ. ID NO. 31, SEQ. ID NO. 32, SEQ. ID NO. 33, SEQ. ID NO. 34, SEQ. ID NO. 35, SEQ. ID NO. 36, SEQ. ID NO. 37, SEQ. ID NO. 38, SEQ. ID NO. 39, SEQ. ID NO. 40, SEQ. ID NO. 41, SEQ. ID NO. 42, SEQ. ID NO. 43, SEQ. ID NO. 44, SEQ. ID NO. 45, SEQ. ID NO. 46, SEQ. ID NO. 47, SEQ. ID NO. 48, SEQ. ID NO. 49, SEQ. ID NO. 50, SEQ. ID NO. 51, SEQ. ID NO. 52, SEQ. ID NO. 53, SEQ. ID NO. 54, SEQ. ID NO. 55, SEQ. ID NO. 56, SEQ. ID NO. 57, SEQ. ID NO. 58, SEQ. ID NO. 59, SEQ. ID NO. 60, SEQ. ID NO. 61, SEQ. ID NO. 62, SEQ. ID NO. 63, SEQ. ID NO. 64, SEQ. ID NO. 65, SEQ. ID NO. 66, SEQ. ID NO. 67, SEQ. ID NO. 68, SEQ. ID NO. 69, SEQ. ID NO. 70, SEQ. ID NO. 71, SEQ. ID NO. 72, SEQ. ID NO. 73, SEQ. ID NO. 74, SEQ. ID NO. 75, SEQ. ID NO. 76, SEQ. ID NO. 77, SEQ. ID NO. 78, SEQ. ID NO. 79, SEQ. ID NO. 80, SEQ. ID NO. 81, SEQ. ID NO. 82, SEQ. ID NO. 83, SEQ. ID NO. 84, SEQ. ID NO. 85, SEQ. ID NO. 86, SEQ. ID NO. 87, SEQ. ID NO. 88, SEQ. ID NO. 89, SEQ. ID NO. 90, SEQ. ID NO. 91, SEQ. ID NO. 92, SEQ. ID NO. 93, SEQ. ID NO. 94, SEQ. ID NO. 95, SEQ. ID NO. 96, SEQ. ID NO. 97, SEQ. ID NO. 98, SEQ. ID NO. 99, SEQ. ID NO. 100, SEQ. ID NO. 101, SEQ. ID NO. 102, SEQ. ID NO. 103, SEQ. ID NO. 104, SEQ. ID NO. 105, SEQ. ID NO. 106, SEQ. ID NO. 107, SEQ. ID NO. 108, SEQ. ID NO. 109, SEQ. ID NO. 110, SEQ. ID NO. 111, SEQ. ID NO. 112, SEQ. ID NO. 113, SEQ. ID NO. 114, SEQ. ID NO. 115, SEQ. ID NO. 116, SEQ. ID NO. 117, SEQ. ID NO. 118, SEQ. ID NO. 119, SEQ. ID NO. 120, SEQ. ID NO. 121, SEQ. ID NO. 122, SEQ. ID NO. 123, SEQ. ID NO. 124, SEQ. ID NO. 125, SEQ. ID NO. 126, SEQ. ID NO. 127, SEQ. ID NO. 128, SEQ. ID NO. 129, SEQ. ID NO. 130, SEQ. ID NO. 131, SEQ. ID NO. 132, SEQ. ID NO. 133, SEQ. ID NO. 134, SEQ. ID NO. 135, SEQ. ID NO. 136, SEQ. ID NO. 137, SEQ. ID NO. 138, SEQ. ID NO. 139, SEQ. ID NO. 140, SEQ. ID NO. 141, SEQ. ID NO. 142, SEQ. ID NO. 143, SEQ. ID NO. 144, SEQ. ID NO. 145, SEQ. ID NO. 146, SEQ. ID NO. 147, SEQ. ID NO. 148, SEQ. ID NO. 149, SEQ. ID NO. 150, SEQ. ID NO. 151, SEQ. ID NO. 152, SEQ. ID NO. 153, SEQ. ID NO. 154, SEQ. ID NO. 155, SEQ. ID NO. 156, SEQ. ID NO. 157, SEQ. ID NO. 158, SEQ. ID NO. 159, SEQ. ID NO. 160, SEQ. ID NO. 161, SEQ. ID NO. 162, SEQ. ID NO. 163, SEQ. ID NO. 164, SEQ. ID NO. 165, SEQ. ID NO. 166, SEQ. ID NO. 167, SEQ. ID NO. 168 and SEQ. ID NO. 169.

16. The method of claim 12, wherein the linear lytic peptide is selected from the group consisting of SEQ ID NO. 4, SEQ ID NO. 5, SEQ ID NO. 8, SEQ ID NO. 9, SEQ ID NO. 10, SEQ ID NO. 38, SEQ ID NO. 73, SEQ ID NO. 92, and SEQ ID NO. 97.

17. The method of claim 12, wherein the lytic base unit peptide is selected from the group consisting of SEQ. ID NO. 170, SEQ. ID NO. 171, SEQ. ID NO. 172, SEQ. ID NO. 173, SEQ. ID NO. 174, SEQ. ID NO. 175, SEQ. ID NO. 176, SEQ. ID NO. 177, SEQ. ID NO. 178, SEQ. ID NO. 179, SEQ. ID NO. 180 and SEQ. ID NO. 181.

18. The method of claim 13, wherein the engineered cationic antimicrobial peptide is selected from the group consisting of SEQ. ID NO. 182, SEQ. ID NO. 183, SEQ. ID NO. 184 and SEQ. ID NO. 185.

19. The method of claim 13, wherein the medical device is selected from a fracture fixation system and a tubular structure.

20. The method of claim 19, wherein the fracture fixation system is selected from a nail, a bolt and a screw.

21. The method of claim 19, wherein the tubular structure is selected from an intubation tube, a feeding tube, a endotracheal tube, a catheter, and a shunt.

22. A fracture fixation system, comprising:
at least one intramedullary nail, wherein the nail is covered with a substantially uniform layer of peptide based antimicrobial selected from a linear lytic peptide, a lytic base unit peptide and an engineered cationic antimicrobial peptide; and
at least one cannulated bone screw or bolt, comprising a plurality of apertures at a bore end, wherein the at least one cannulated bone screw or bolt is covered with the substantially uniform layer of peptide based antimicrobial a linear lytic peptide, a lytic base unit peptide and an engineered cationic antimicrobial peptide.

23. The fracture fixation system of claim 23, wherein the fracture fixation system further comprises a composite material comprised of a bone growth promoter and a peptide-based antimicrobial selected from a linear lytic peptide, a lytic based peptide and an engineered cationic antimicrobial peptide; wherein the bone growth promoter is perfused through the plurality of apertures at the bore end.

24. The fracture fixation system of claim 22, wherein the linear lytic peptide is selected from the group consisting of SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15, SEQ. ID NO. 16, SEQ. ID NO. 17, SEQ. ID NO. 18, SEQ. ID NO. 19, SEQ. ID NO. 20, SEQ. ID NO. 21, SEQ. ID NO. 22, SEQ. ID NO. 23, SEQ. ID NO. 24, SEQ. ID NO. 25, SEQ. ID NO. 26, SEQ. ID NO. 27, SEQ. ID NO. 28, SEQ. ID NO. 29, SEQ. ID NO. 30, SEQ. ID NO. 31, SEQ. ID NO. 32, SEQ. ID NO. 33, SEQ. ID NO. 34, SEQ. ID NO. 35, SEQ. ID NO. 36, SEQ. ID NO. 37, SEQ. ID NO. 38, SEQ. ID NO. 39, SEQ. ID NO. 40, SEQ. ID NO. 41, SEQ. ID NO. 42, SEQ. ID NO. 43, SEQ. ID NO. 44, SEQ. ID NO. 45, SEQ. ID NO. 46, SEQ. ID NO. 47, SEQ. ID NO. 48, SEQ. ID NO. 49, SEQ. ID NO. 50, SEQ. ID NO. 51, SEQ. ID NO. 52, SEQ. ID NO. 53, SEQ. ID NO. 54, SEQ. ID NO. 55, SEQ. ID NO. 56, SEQ. ID NO. 57, SEQ. ID NO. 58, SEQ. ID NO. 59, SEQ. ID NO. 60, SEQ. ID NO. 61, SEQ. ID NO. 62, SEQ. ID NO. 63, SEQ. ID NO. 64, SEQ. ID NO. 65, SEQ. ID NO. 66, SEQ. ID NO. 67, SEQ. ID NO. 68, SEQ. ID NO. 69, SEQ. ID NO. 70, SEQ. ID NO. 71, SEQ. ID NO. 72, SEQ. ID NO. 73, SEQ. ID NO. 74, SEQ. ID NO. 75, SEQ. ID NO. 76, SEQ. ID NO. 77, SEQ. ID NO. 78, SEQ. ID NO. 79, SEQ. ID NO. 80, SEQ. ID NO. 81, SEQ. ID NO. 82, SEQ. ID NO. 83, SEQ. ID NO. 84, SEQ. ID NO. 85, SEQ. ID NO. 86, SEQ. ID NO. 87, SEQ. ID NO. 88, SEQ. ID NO. 89, SEQ. ID NO. 90, SEQ. ID NO. 91, SEQ. ID NO. 92, SEQ. ID NO. 93, SEQ. ID NO. 94, SEQ. ID NO. 95, SEQ. ID NO. 96, SEQ. ID NO. 97, SEQ. ID NO. 98, SEQ. ID NO. 99, SEQ. ID NO. 100, SEQ. ID NO. 101, SEQ. ID NO. 102, SEQ. ID NO. 103, SEQ. ID NO. 104, SEQ. ID NO. 105, SEQ. ID NO. 106, SEQ. ID NO. 107, SEQ. ID NO. 108, SEQ. ID NO. 109, SEQ. ID NO. 110, SEQ. ID NO. 111, SEQ. ID NO. 112, SEQ. ID NO. 113, SEQ. ID NO. 114, SEQ. ID NO. 115, SEQ. ID NO. 116, SEQ. ID NO. 117, SEQ. ID NO. 118, SEQ. ID NO. 119, SEQ. ID NO. 120, SEQ. ID NO. 121, SEQ. ID NO. 122, SEQ. ID NO. 123, SEQ. ID NO. 124, SEQ. ID NO. 125, SEQ. ID NO. 126, SEQ. ID NO. 127, SEQ. ID NO. 128, SEQ. ID NO. 129, SEQ. ID NO. 130, SEQ. ID NO. 131, SEQ. ID NO. 132, SEQ. ID NO. 133, SEQ. ID NO. 134, SEQ. ID NO. 135, SEQ. ID NO. 136, SEQ. ID NO. 137, SEQ. ID NO. 138, SEQ. ID NO. 139, SEQ. ID NO. 140, SEQ. ID NO. 141, SEQ. ID NO. 142, SEQ. ID NO. 143, SEQ. ID NO. 144, SEQ. ID NO. 145, SEQ. ID NO. 146, SEQ. ID NO. 147, SEQ. ID NO. 148, SEQ. ID NO. 149, SEQ. ID NO. 150, SEQ. ID NO. 151, SEQ. ID NO. 152, SEQ. ID NO. 153, SEQ. ID NO. 154, SEQ. ID NO. 155, SEQ. ID NO. 156, SEQ. ID NO. 157, SEQ. ID NO. 158, SEQ. ID NO. 159, SEQ. ID NO. 160, SEQ. ID NO. 161, SEQ. ID NO. 162, SEQ. ID NO. 163, SEQ. ID NO. 164, SEQ. ID NO. 165, SEQ. ID NO. 166, SEQ. ID NO. 167, SEQ. ID NO. 168 and SEQ. ID NO. 169.

25. The fracture fixation system of claim 22, wherein the linear lytic peptide is selected from the group consisting of SEQ ID NO. 4, SEQ ID NO. 5, SEQ ID NO. 8, SEQ ID NO. 9, SEQ ID NO. 10, SEQ ID NO. 38, SEQ ID NO. 73, SEQ ID NO. 92, and SEQ ID NO. 97.

26. The fracture fixation system of claim 22, wherein the lytic base unit peptide is selected from the group consisting of SEQ. ID NO. 170, SEQ. ID NO. 171, SEQ. ID NO. 172, SEQ. ID NO. 173, SEQ. ID NO. 174, SEQ. ID NO. 175, SEQ. ID NO. 176, SEQ. ID NO. 177, SEQ. ID NO. 178, SEQ. ID NO. 179, SEQ. ID NO. 180 and SEQ. ID NO. 181.

27. The fracture fixation system of claim 22, wherein the engineered cationic antimicrobial peptide is selected from the group consisting of SEQ. ID NO. 182, SEQ. ID NO. 183, SEQ. ID NO. 184 and SEQ. ID NO. 185.

28. An intubation system comprising:
at least one tube, wherein the at least one tube comprises an internal surface and an external surface that are coated substantially uniformly with an antimicrobial peptide selected from a linear lytic peptide, a lytic base unit peptide and an engineered cationic antimicrobial peptide;
at least one cuff, wherein the at least one cuff comprises an internal surface and an external surface that are coated substantially uniformly with an antimicrobial peptide selected from a linear lytic peptide, a lytic base unit peptide and an engineered cationic antimicrobial peptide; and
at least one tip, wherein the at least one tip comprises an internal surface and an external surface that are coated substantially uniformly with an antimicrobial peptide selected from a linear lytic peptide, a lytic base unit peptide and an engineered cationic antimicrobial peptide.

29. The intubation system of claim 28, wherein the linear lytic peptide is selected from the group consisting of SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15, SEQ. ID NO. 16, SEQ. ID NO. 17, SEQ. ID NO. 18, SEQ. ID NO. 19, SEQ. ID NO. 20, SEQ. ID NO. 21, SEQ. ID NO. 22, SEQ. ID NO. 23, SEQ. ID NO. 24, SEQ. ID NO. 25, SEQ. ID NO. 26, SEQ. ID NO. 27, SEQ. ID NO. 28, SEQ. ID NO. 29, SEQ. ID NO. 30, SEQ. ID NO. 31, SEQ. ID NO. 32, SEQ. ID NO. 33, SEQ. ID NO. 34, SEQ. ID NO. 35, SEQ. ID NO. 36, SEQ. ID NO. 37, SEQ. ID NO. 38, SEQ. ID NO. 39, SEQ. ID NO. 40, SEQ. ID NO. 41, SEQ. ID NO. 42, SEQ. ID NO. 43, SEQ. ID NO. 44, SEQ. ID NO. 45, SEQ. ID NO. 46, SEQ. ID NO. 47, SEQ. ID NO. 48, SEQ. ID NO. 49, SEQ. ID NO. 50, SEQ. ID NO. 51, SEQ. ID NO. 52, SEQ. ID NO. 53, SEQ. ID NO. 54, SEQ. ID NO. 55, SEQ. ID NO. 56, SEQ. ID NO. 57, SEQ. ID NO. 58, SEQ. ID NO. 59, SEQ. ID NO. 60, SEQ. ID NO. 61, SEQ. ID NO. 62, SEQ. ID NO. 63, SEQ. ID NO. 64, SEQ. ID NO. 65, SEQ. ID NO. 66, SEQ. ID NO. 67, SEQ. ID NO. 68, SEQ. ID NO. 69, SEQ. ID NO. 70, SEQ. ID NO. 71, SEQ. ID NO. 72, SEQ. ID NO. 73, SEQ. ID NO. 74, SEQ. ID NO. 75, SEQ. ID NO. 76, SEQ. ID NO. 77, SEQ. ID NO. 78, SEQ. ID NO. 79, SEQ. ID NO. 80, SEQ. ID NO. 81, SEQ. ID NO. 82, SEQ. ID NO. 83, SEQ. ID NO. 84, SEQ. ID NO. 85, SEQ. ID NO. 86, SEQ. ID NO. 87, SEQ. ID NO. 88, SEQ. ID NO. 89, SEQ. ID NO. 90, SEQ. ID NO. 91, SEQ. ID NO. 92, SEQ. ID NO. 93, SEQ. ID NO. 94, SEQ. ID NO. 95, SEQ. ID NO. 96, SEQ. ID NO. 97, SEQ. ID NO. 98, SEQ. ID NO. 99, SEQ. ID NO. 100, SEQ. ID NO. 101, SEQ. ID NO. 102, SEQ. ID NO. 103, SEQ. ID NO. 104, SEQ. ID NO. 105, SEQ. ID NO. 106, SEQ. ID NO. 107, SEQ. ID NO. 108, SEQ. ID NO. 109, SEQ. ID NO. 110, SEQ. ID NO. 111, SEQ. ID NO. 112, SEQ. ID NO. 113, SEQ. ID NO. 114, SEQ. ID NO. 115, SEQ. ID NO. 116, SEQ. ID NO. 117, SEQ. ID NO. 118, SEQ. ID NO. 119, SEQ. ID NO. 120, SEQ. ID NO. 121, SEQ. ID NO. 122, SEQ. ID NO. 123, SEQ. ID NO. 124, SEQ. ID NO. 125, SEQ. ID NO. 126, SEQ. ID NO. 127, SEQ. ID NO. 128, SEQ. ID NO. 129, SEQ. ID NO. 130, SEQ. ID NO. 131, SEQ. ID NO. 132, SEQ. ID NO. 133, SEQ. ID NO. 134, SEQ. ID NO. 135, SEQ. ID NO. 136, SEQ. ID NO. 137, SEQ. ID NO. 138, SEQ. ID NO. 139, SEQ. ID NO. 140, SEQ. ID NO. 141, SEQ. ID NO. 142, SEQ. ID NO. 143, SEQ. ID NO. 144, SEQ. ID NO. 145, SEQ. ID NO. 146, SEQ. ID NO. 147, SEQ. ID NO. 148, SEQ. ID NO. 149, SEQ. ID NO. 150, SEQ. ID NO. 151, SEQ. ID NO. 152, SEQ. ID NO. 153, SEQ. ID NO. 154, SEQ. ID NO. 155, SEQ. ID NO. 156, SEQ. ID NO. 157, SEQ. ID NO. 158, SEQ. ID NO. 159, SEQ. ID NO. 160, SEQ. ID NO. 161, SEQ. ID NO. 162, SEQ. ID NO. 163, SEQ. ID NO. 164, SEQ. ID NO. 165, SEQ. ID NO. 166, SEQ. ID NO. 167, SEQ. ID NO. 168 and SEQ. ID NO. 169.

30. The intubation system of claim 28, wherein the linear lytic peptide is selected from the group consisting of SEQ ID NO. 4, SEQ ID NO. 5, SEQ ID NO. 8, SEQ ID NO. 9, SEQ ID NO. 10, SEQ ID NO. 38, SEQ ID NO. 73, SEQ ID NO. 92, and SEQ ID NO. 97.

31. The intubation system of claim 28, wherein the lytic base unit peptide is selected from the group consisting of SEQ. ID NO. 170, SEQ. ID NO. 171, SEQ. ID NO. 172, SEQ. ID NO. 173, SEQ. ID NO. 174, SEQ. ID NO. 175, SEQ. ID NO. 176, SEQ. ID NO. 177, SEQ. ID NO. 178, SEQ. ID NO. 179, SEQ. ID NO. 180 and SEQ. ID NO. 181.

32. The intubation system of claim 28, wherein the engineered cationic antimicrobial peptide is selected from the group consisting of SEQ. ID NO. 182, SEQ. ID NO. 183, SEQ. ID NO. 184, SEQ. ID NO. 185.

33. A method of preventing infection in a subject, comprising:
coating at least a portion of a surface of a medical device with a therapeutic amount of an antimicrobial peptide selected from a linear lytic peptide, a lytic base unit peptide and an engineered cationic antimicrobial peptide; and
implanting the coated medical device in the subject.

34. The method of claim 33, wherein the linear lytic peptide is selected from the group consisting of SEQ. ID NO. 1, SEQ. ID NO. 2, SEQ. ID NO. 3, SEQ. ID NO. 4, SEQ. ID NO. 5, SEQ. ID NO. 6, SEQ. ID NO. 7, SEQ. ID NO. 8, SEQ. ID NO. 9, SEQ. ID NO. 10, SEQ. ID NO. 11, SEQ. ID NO. 12, SEQ. ID NO. 13, SEQ. ID NO. 14, SEQ. ID NO. 15, SEQ. ID NO. 16, SEQ. ID NO. 17, SEQ. ID NO. 18, SEQ. ID NO. 19, SEQ. ID NO. 20, SEQ. ID NO. 21, SEQ. ID NO. 22, SEQ. ID NO. 23, SEQ. ID NO. 24, SEQ. ID NO. 25, SEQ. ID NO. 26, SEQ. ID NO. 27, SEQ. ID NO. 28, SEQ. ID NO. 29, SEQ. ID NO. 30, SEQ. ID NO. 31, SEQ. ID NO. 32, SEQ. ID NO. 33, SEQ. ID NO. 34, SEQ. ID NO. 35, SEQ. ID NO. 36, SEQ. ID NO. 37, SEQ. ID NO. 38, SEQ. ID NO. 39, SEQ. ID NO. 40, SEQ. ID NO. 41, SEQ. ID NO. 42, SEQ. ID NO. 43, SEQ. ID NO. 44, SEQ. ID NO. 45, SEQ. ID NO. 46, SEQ. ID NO. 47, SEQ. ID NO. 48, SEQ. ID NO. 49, SEQ. ID NO. 50, SEQ. ID NO. 51, SEQ. ID NO. 52, SEQ. ID NO. 53, SEQ. ID NO. 54, SEQ. ID NO. 55, SEQ. ID NO. 56, SEQ. ID NO. 57, SEQ. ID NO. 58, SEQ. ID NO. 59, SEQ. ID NO. 60, SEQ. ID NO. 61, SEQ. ID NO. 62, SEQ. ID NO. 63, SEQ. ID NO. 64, SEQ. ID NO. 65, SEQ. ID NO. 66, SEQ. ID NO. 67, SEQ. ID NO. 68, SEQ. ID NO. 69, SEQ. ID NO. 70, SEQ. ID NO. 71, SEQ. ID NO. 72, SEQ. ID NO. 73, SEQ. ID NO. 74, SEQ. ID NO. 75, SEQ. ID NO. 76, SEQ. ID NO. 77, SEQ. ID NO. 78, SEQ. ID NO. 79, SEQ. ID NO. 80, SEQ. ID NO. 81, SEQ. ID NO. 82, SEQ. ID NO. 83, SEQ. ID NO. 84, SEQ. ID NO. 85, SEQ. ID NO. 86, SEQ. ID NO. 87, SEQ. ID NO. 88, SEQ. ID NO. 89, SEQ. ID NO. 90, SEQ. ID NO. 91, SEQ. ID NO. 92, SEQ. ID NO. 93, SEQ. ID NO. 94, SEQ. ID NO. 95, SEQ. ID NO. 96, SEQ. ID NO. 97, SEQ. ID NO. 98, SEQ. ID NO. 99, SEQ. ID NO. 100, SEQ. ID NO. 101, SEQ. ID NO. 102, SEQ. ID NO. 103, SEQ. ID NO. 104, SEQ. ID NO. 105, SEQ. ID NO. 106, SEQ. ID NO. 107, SEQ. ID NO. 108, SEQ. ID NO. 109, SEQ. ID NO. 110, SEQ. ID NO. 111, SEQ. ID NO. 112, SEQ. ID NO. 113, SEQ. ID NO. 114, SEQ. ID NO. 115, SEQ. ID NO. 116, SEQ. ID NO. 117, SEQ. ID NO. 118, SEQ. ID NO. 119, SEQ. ID NO. 120, SEQ. ID NO. 121, SEQ. ID NO. 122, SEQ. ID NO. 123, SEQ. ID NO. 124, SEQ. ID NO. 125, SEQ. ID NO. 126, SEQ. ID NO. 127, SEQ. ID NO. 128, SEQ. ID NO. 129, SEQ. ID NO. 130, SEQ. ID NO. 131, SEQ. ID NO. 132, SEQ. ID NO. 133, SEQ. ID NO. 134, SEQ. ID NO. 135, SEQ. ID NO. 136, SEQ. ID NO. 137, SEQ. ID NO. 138, SEQ. ID NO. 139, SEQ. ID NO. 140, SEQ. ID NO. 141, SEQ. ID NO. 142, SEQ. ID NO. 143, SEQ. ID NO. 144, SEQ. ID NO. 145, SEQ. ID NO. 146, SEQ. ID NO. 147, SEQ. ID NO. 148, SEQ. ID NO. 149, SEQ. ID NO. 150, SEQ. ID NO. 151, SEQ. ID NO. 152, SEQ. ID NO. 153, SEQ. ID NO. 154, SEQ. ID NO. 155, SEQ. ID NO. 156, SEQ. ID NO. 157, SEQ. ID NO. 158, SEQ. ID NO. 159, SEQ. ID NO. 160, SEQ. ID NO. 161, SEQ. ID NO. 162, SEQ. ID NO. 163, SEQ. ID NO. 164, SEQ. ID NO. 165, SEQ. ID NO. 166, SEQ. ID NO. 167, SEQ. ID NO. 168 and SEQ. ID NO. 169

35. The method of claim 33, wherein the linear lytic peptide is selected from the group consisting of SEQ ID NO. 4, SEQ ID NO. 5, SEQ ID NO. 8, SEQ ID NO. 9, SEQ ID NO. 10, SEQ ID NO. 38, SEQ ID NO. 73, SEQ ID NO. 92, and SEQ ID NO. 97.

36. The method of claim 33, wherein the lytic base unit peptide is selected from the group consisting of SEQ. ID NO. 170, SEQ. ID NO. 171, SEQ. ID NO. 172, SEQ. ID NO. 173, SEQ. ID NO. 174, SEQ. ID NO. 175, SEQ. ID NO. 176, SEQ. ID NO. 177, SEQ. ID NO. 178, SEQ. ID NO. 179, SEQ. ID NO. 180 and SEQ. ID NO. 181.

37. The method of claim 33, wherein the engineered cationic antimicrobial peptide is selected from the group consisting of SEQ. ID NO. 182, SEQ. ID NO. 183, SEQ. ID NO. 184 and SEQ. ID NO. 185.

38. The method of claim 33, wherein the medical device is selected from a fracture fixation system, a tubular device that penetrates a body tissue of a patient, and a component of an intubation system.

39. The method of claim 38, wherein the fracture fixation system is selected from a nail, a bolt and a screw.

40. The method of claim 38, wherein the tubular structure is selected from an intubation tube, a feeding tube, a endotracheal tube, a catheter, and a shunt.
